(19) United States
(12) Patent Application Publication
(10) Pub. No.: US 2026/0245673 A1
(43) Pub. Date: Aug. 20, 2026

(54) FRAGRANCE AND FLAVOUR GENERATION

(71) Applicant: GIVAUDAN SA, VERNIER (CH)

(72) Inventors: Stephen NILSEN, New York, NY (US); Valerie DROBAC, VERNIER (CH); Cedric PARIS, Lausanne (CH)

(21) Appl. No.: 19/159,811

(22) PCT Filed: Feb. 28, 2024
(86) PCT No.: PCT/EP2024/055046

§ 371 Date: Aug. 26, 2025

Related U.S. Application Data
(60) Provisional application No. 63/448,805, filed on Feb. 28, 2023.

(51) Int. Cl. G16C 20/70; G16C 60/00

(52) U.S. Cl. CPC G16C 20/70; G16C 60/00

(57) ABSTRACT

A computer-implemented method of training a system for generating a formula of ingredients for a fragrance, the method comprising: receiving an input dataset comprising ingredient palettes of known fragrances and/or flavours and ingredient concentrations; training a palette generation machine learning model using the input dataset, wherein the palette generation machine learning model, when trained, is configured to generate at least one generated ingredient palette; and training a concentration generation machine learning model using the input dataset, wherein the concentration generation machine learning model, when trained, is configured to generate at least one generated formula, each generated formula comprising ingredient concentrations for a generated ingredient palette.

### FIELD OF THE INVENTION

The present invention relates to fragrances and flavours, described by a palette of ingredients and associated ingredient concentration. More particularly, the present invention relates to a method of training machine learning (ML) models and a method of use of ML models for use in the generation of fragrances and/or flavours, as well as to a data processing apparatus, a computer program, and a computer-readable storage medium for carrying out said methods.

### BACKGROUND OF THE INVENTION

In the technical area of perfume design, the generation of formulae (including both fragrances and flavours), including ingredient palettes and ingredient concentrations, is a highly skilled art. For example, state of the art fragrances are prepared using a palette of fragrance ingredients. At the heart of the formulae generation process lies the skill of the perfumer in making inspired combinations from the ingredient palette. However, modern technologies are increasingly used to aid in the creative process. For example, ingredient palettes may be stored on a computer database, allowing formulae to be created and displayed on computer interfaces.

A formula may be displayed on a computer interface in the form of a list of the names and quantities or concentrations of the relevant ingredients. Based on the olfactive and/or flavour character of each ingredient, as well as the relative proportions in which they are employed, an experienced perfumer or flavourist may be able to form a reasonable mental impression of the odour and/or the flavour of the fragrance, which will be of some assistance in guiding them through the creation process.

Once a formula is finalised, a digital signal expressing the recipe may be sent to an output device, which may be adapted to mix and dispense (compound) a fragrance or flavour for further olfactive or flavour assessment.

However, even for very experienced perfumers and flavourists, the creative process may entail multiple iterations of formula adjustment in which complex creations require multiple design, dispensing, and olfactive or flavour assessment steps, all of which are time consuming, exhausting on the nose and the palate of the perfumer or flavourist, and wasteful of valuable fragrance and flavour ingredients.

There is therefore a need for techniques that enable perfumers, flavourists, evaluators, and any other actors involved in the formula generation process to simplify the formula generation process.

### SUMMARY OF THE INVENTION

The invention is defined in the independent claims, to which reference should now be made. Further features are set out in the dependent claims.

According to an aspect of the invention, there is provided a method of training a system for generating a formula (or a collection of formulae) of ingredients for a generated fragrance and/or for a generated flavour. The generated formula is suitable for review by a human or computational actor or may be directly produced. The method includes receiving an input dataset, the input dataset including a plurality of ingredient palettes of known fragrances and/or flavours, with one ingredient palette per known fragrance and/or flavour. The input dataset also includes ingredient concentrations of ingredients in the palette for each known fragrance and/or flavour.

The method also includes training a palette generation ML model (a first ML model) using at least a portion of the input dataset. The palette generation ML model, when trained, is configured to generate at least one potential or prospective generated ingredient palette.

The method also includes training a concentration generation ML model (a second ML model) using at least a portion of the input dataset. The concentration generation ML model, when trained, is configured to generate at least one potential or prospective generated formula. Each generated formula includes ingredient concentrations for one generated ingredient palette, as generated by the palette generation ML model. In this way, the concentration generation ML model may be thought of as “filling-in” the ingredient concentrations for each generated palette of ingredients.

Thus, the system, when trained, comprises a trained palette generation ML model and a trained concentration generation ML model.

Optionally, the input dataset includes end-uses for each of the known fragrances and/or flavours. This allows either (or both) the palette generation ML model or the concentration generation ML model to be trained with the conditional parameter of “end-use”. In turn, the trained models may be configured to output palettes and concentrations tailored for specific end-uses. The input dataset may therefore further include a specified end-use, enabling the trained models to generate palettes and/or concentrations for such specified end-use.

Optionally, the method causes a computer to accept input of a specified end-use, where the specified end-use may be limited to the end-uses of the known fragrances and/or flavours in the input dataset. The method then re-trains or fine-tunes the palette generation ML model. Retraining occurs using the ingredient palettes and ingredient concentrations of the known fragrances and/or flavours associated with the specified end-use. The retrained palette generation ML model (or models, with one model for each end-use) generates ingredient palettes for the specified end-use. Training the concentration generation ML model then uses ingredient palettes for the specified end-use. As the original task (training the palette generation ML model) is similar to this new task (retraining the palette generation ML model), the retraining process enables accurate generation of palettes for specific end-uses, without the need to acquire a large dataset specifically for that specific end-use. Rather, the full dataset comprising fragrances for all end-uses may be suitable for teaching the models learned features applicable to all end-uses and the narrower end-use-specified dataset then refines these teachings.

Optionally, the palette generation ML model (or, indeed, the retrained palette generation ML model(s)) is a variational autoencoder, VAE. Optionally, the VAE is a conditional VAE, CVAE, where some condition (e.g., end-use or author of known fragrances/flavours) may be imposed throughout training so as to enforce generation of palettes in accordance with the condition. Alternatively, the palette generation ML model (or retrained palette generation ML model) may be a (first) generative adversarial network, GAN. Again, some condition may be imposed with this GAN.

Optionally, the concentration generation ML model may be a bucket predictor, BP. Alternatively, the concentration generation ML model may be a (second) GAN.

Optionally, for instance when the palette generation ML model is a CVAE, the palette generation ML model may—when trained—output scores (e.g., probabilities) related to the presence of each ingredient within each generated ingredient palette. For each generated ingredient palette (and for each ingredient within that palette), the method may determine that an ingredient is to be included within the palette when the score of the presence of that ingredient exceed some predetermined threshold. The predetermined threshold may be based on the proportional presence of that ingredient in the ingredient palettes of the known fragrances and/or flavours in the input dataset. In this way, ingredients that are rarely used in known fragrances and/or flavours appear with a similar score in generated formulae. Without such modification, where a static threshold (e.g., 0.5) is used, there is a chance that rarely used ingredients appear in no generated formulae.

Optionally, where the input dataset includes end-uses of the known fragrances and/or flavours and where the palette generation ML model is a VAE comprising an encoder-decoder architecture, the training method may involve passing the end-use of the known fragrances and/or flavours to a plurality of layers (optionally all layers) in the encoder. Similarly, the method may involve passing the end-use to a plurality of layers (optionally all layers) in the decoder.

In this way, the condition (end-use) may be concatenated to the output data at each relevant layer and the complete model may be trained with the “intent” that the model learns the relationships between inputs with the same end-use. That is, condition concatenation reinforces the end-use information when training. Similarly, condition concatenation may be used for palette generation ML models in the form of GANs, where the end-use is reinforced at multiple layers of the underlying network. Condition concatenation may also be used in trained models, to reinforce the end-use when performing inference.

Optionally, the concentration generation ML model may too implement condition concatenation. For instance, the concentration generation ML model may comprise an encoder-decoder architecture and the condition (end-use) may be reinforced as described above. Similarly, the concentration generation ML model may be in the form of a GAN, and the end-use may be reinforced at multiple layers of the underlying network.

Optionally, where the palette generation ML model is a VAE comprising multiple layers, the training method may involve—for each training mini-batch of the input dataset, and for each layer of the CVAE—normalising the output for each layer. This ensures that the input to each layer is stable, thus ensuring the training process is efficient.

Optionally, the method may include, following training (and/or retraining) of both palette generation ML model and concentration generation ML model, running the models so as to generate at least one generated formula. Thus, the trained system allows for generation of prospective formulae without the need to manually put together ingredient palettes and ingredient concentrations, a task that is typically possible only by experienced perfumers.

Optionally, following generation of at least one formula, the method involves quantifying each formula by its expected odour or olfactive characteristics.

According to another aspect of the invention, there is a provided a method of generating a formula (or a collection of formulae) of ingredients for a generated fragrance. The method includes running a trained (or retrained) palette generation ML model to generate at least one potential (or prospective or candidate) generated ingredient palette. The palette generation ML model may be trained in accordance with other aspects of the invention.

The method also includes running a trained concentration generation ML model to generate at least one potential (generated) formula, each generated formula including ingredient concentrations for one of the generated ingredient palettes. The concentration generation ML model may be trained in accordance with other aspects of the invention.

Optionally, following generation of at least one generated formula, the method may comprise generating instructions for manufacture of a formula in accordance with the generated ingredient palette and associated generated concentrations. Instructions may be in the form of instructions for a human user (including, e.g., quantities of each ingredient to compound) or may be instructions in the form of machine instructions, suitable for transmission (via wire or wirelessly) to a machine to cause manufacture of the formula.

Optionally, the method may also cause a computer to provide a user interface. The user interface may be suitable for displaying at least a subset of generated formulae. The user interface may be configured to accept user input, enabling the computer to filter or select a subset of the generated formulae for display. In this way, the user may filter a large, generated dataset to only formulae of relevance or interest.

Optionally, the method may enable user input (into the user interface) to cause manufacture of a formula. The user interface provides an alternative graphical shortcut, allowing the user to directly set manufacturing conditions without need for manual input of, e.g., specific ingredients and concentrations.

According to another aspect of the invention, there is provided a method of generating a formula, the method comprising a training method for training ML models in accordance with other aspects of the invention and comprising a formula generation method using the trained ML models in accordance with other aspects of the invention.

An apparatus (computer or computer system) or computer program according to preferred embodiments may comprise any combination of the method aspects. Methods or computer programs according to further embodiments may be described as computer-implemented in that they require processing and memory capability.

The apparatus according to preferred embodiments is described as configured or arranged to, or simply “to” carry out certain functions. This configuration or arrangement could be by use of hardware or middleware or any other suitable system. In preferred embodiments, the configuration or arrangement is by software.

Thus, according to one aspect there is provided a program which, when loaded onto at least one computer configures the computer to become the apparatus according to any of the preceding apparatus definitions or any combination thereof.

In general the computer may comprise the elements listed as being configured or arranged to provide the functions defined. For example this computer may include memory, processing, and a network interface, as well as an input device.

The invention may be implemented in digital electronic circuitry, or in computer hardware, firmware, software, or in combinations of them. The invention may be implemented as a computer program or computer program product, i.e., a computer program tangibly embodied in a non-transitory information carrier, e.g., in a machine-readable storage device, or in a propagated signal, for execution by, or to control the operation of, one or more hardware modules.

A computer program may be in the form of a stand-alone program, a computer program portion, or more than one computer program and may be written in any form of programming language, including compiled or interpreted languages, and it may be deployed in any form, including as a stand-alone program or as a module, component, subroutine, or other unit suitable for use in a data processing environment. A computer program may be deployed to be executed on one module or on multiple modules at one site or distributed across multiple sites and interconnected by a communication network.

Method steps of the invention may be performed by one or more programmable processors executing a computer program to perform functions of the invention by operating on input data and generating output. Apparatus of the invention may be implemented as programmed hardware or as special purpose logic circuitry, including e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions coupled to one or more memory devices for storing instructions and data.

The invention is described in terms of particular embodiments. Other embodiments are within the scope of the following claims. For example, the steps of the invention may be performed in a different order and still achieve desirable results. Multiple test script versions may be edited and invoked as a unit without using object-oriented programming technology; for example, the elements of a script object may be organized in a structured database or a file system, and the operations described as being performed by the script object may be performed by a test control program.

Elements of the invention have been described using the terms “processor”, “input device” etc. The skilled person will appreciate that such functional terms and their equivalents may refer to parts of the system that are spatially separate but combine to serve the function defined. Equally, the same physical parts of the system may provide two or more of the functions defined. For example, separately defined means may be implemented using the same memory and/or processor as appropriate.

### BRIEF DESCRIPTION OF THE DRAWINGS

Reference is made, by way of example only, to the accompanying drawings in which:

FIG. 1 is a flow chart of a method of training a system for generating a formula of ingredients for a fragrance, according to an embodiment;

FIG. 2 is a flow chart of a method of generating a formula of ingredients for a fragrance, according to an embodiment;

FIG. 3 is a schematic diagram of a CVAE, for use in the generation of a palette;

FIG. 4 is a schematic diagram of a GAN, for use in the generation of a palette;

FIG. 5 is another schematic diagram of a GAN, for use in the generation of a palette;

FIG. 6 is a schematic diagram of a BP, for use in the generation of concentrations;

FIG. 7 is a schematic diagram of a GAN, for use in the generation of concentrations;

FIG. 8 is a schematic diagram of condition (end-use) concatenation, applied to a CVAE for use in the generation of a palette;

FIG. 9 is a schematic diagram of the training process for a GAN, for use—when trained—in the generation of a palette;

FIG. 10 is a further schematic diagram of the training process for a GAN, for use—when trained—in concentration generation;

FIG. 11 is a diagram demonstrating underlying principles of a palette evaluation technique using a linear combination/convex embedding approach;

FIG. 12 is a box plot of the number of ingredients for CVAE-generated palettes across distinct end-uses (labelled A to Z), with a frequency threshold modification;

FIG. 13 is a box plot of the number of ingredients for CVAE-generated palettes across distinct end-use, with a frequency threshold modification, a condition concatenation modification, and a batch normalization modification;

FIG. 14 is a two-dimensional KDE plot for the two principal components found for generated palette embeddings;

FIG. 15 is a two-dimensional KDE plot for the two principal components found for generated embeddings, using a loss function-modified LDA with ingredient proportion pre-processing;

FIG. 16 is a diagram illustrating pre-processing of a BOM, for olfactive description determination;

FIG. 17 is a diagram illustrating recasting ingredient concentration into intensity, for olfactive description determination;

FIG. 18 is a diagram of an example ingredient representation, for olfactive description determination;

FIG. 19 is a diagram illustrating the representation of an example ingredient (rose absolute incolore DM) as a path, for olfactive description determination;

FIG. 20 is a diagram demonstrating the projection of an example formula into classification trees, for olfactive description determination;

FIG. 21 is a diagram illustrating the computation of formula similarity, for each layer of classification, for olfactive description determination;

FIG. 22 is a diagram illustrating computation of the weighted sum of all layers, for olfactive description determination;

FIG. 23 is a diagram illustrating clustering of formulae, for olfactive description determination;

FIG. 24 is a pair of diagrams demonstrating the olfactive properties of an example formula;

FIG. 25 is an example user interface, suitable for provision to a user to enable filtering of generated formulae;

FIG. 26 is a further example user interface, illustrating the results of filtering;

FIG. 27 is a further example user interface, illustrating further results of filtering;

FIG. 28 is another example user interface, illustrating the results of a distinct filtering process; and

FIG. 29 is a diagram of suitable hardware for implementation of embodiments.

### DETAILED DESCRIPTION

The inventors have come to the realisation that artificial intelligence techniques are well-suited for addressing the shortfalls associated with protracted trial and error techniques, which rely on the availability of highly skilled perfumers and flavourists.

Generative ML algorithms and associated ML models are suited for generation of large quantities of prospective fragrances. The inventors have found that decomposition of the task of generating fragrances into constituent tasks of generating ingredient palates and generating ingredient concentrations reliably generates large numbers of fragrances that experts deem to be suitable for manufacture.

Moreover, the generation of prospective formulae using embodiments is “guided” by pre-existing knowledge and expertise captured within training datasets. In this way, prospective formulae may capture traits, habits, and preferences of experienced perfumers and flavourists, which the user may not necessary have otherwise considered.

FIG. 1 is a flow chart of a method of training a system for generating a formula (or formulae) of ingredients for a generated fragrance (or fragrances), for user review.

S10 sees the computer receiving an input dataset, the input dataset including ingredient palettes of known fragrances and/or flavours. The input dataset further includes concentrations (e.g., in wt. %, vol. %, or mol. %) of ingredients in the palette, for each known fragrance and/or flavour. Optionally, the input dataset further includes end-use (or end-uses if multiple end-uses are relevant) for each known fragrance and/or flavour.

S12 causes the computer to train a palette generation ML model using the input dataset. The palette generation ML model, when trained, is configured to generate a plurality of potential or prospective generated ingredient palettes. Optionally, these may be generated for a specified end-use.

S14 causes the computer to train a concentration generation ML model using the input dataset. Optionally, the training data here may be concatenated with one or more conditional variables, such as the specified end-use. The concentration generation ML model, when trained, is configured to generate at least one potential or prospective generated formula. Each generated formula comprises ingredient concentrations for one of the generated ingredient palettes.

In an example, palette generation ML models and concentration ML models are trained on on-premise development platforms. Example trained ML models are hosted on AWS cloud services, in an S3 bucket. Example trained ML models are saved as pickle files.

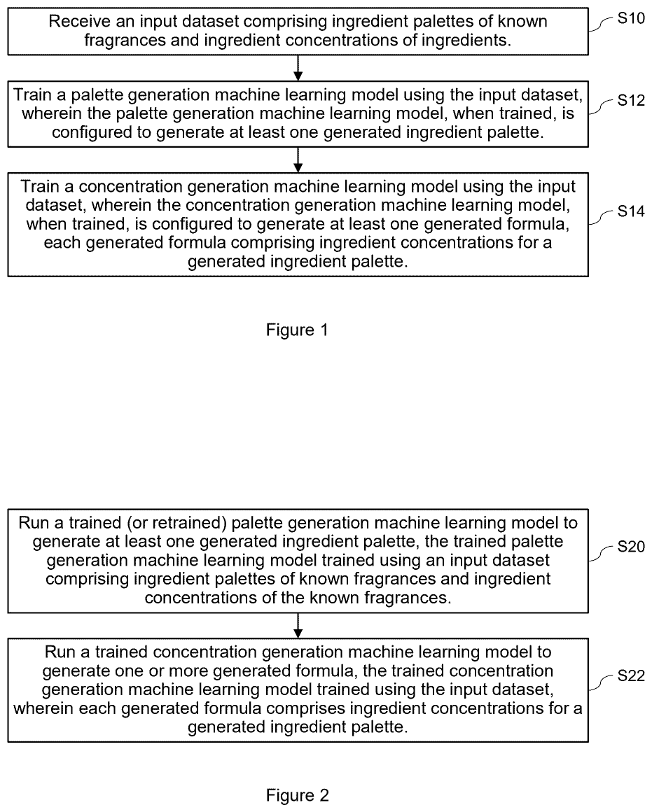

FIG. 2 is a flow chart of a method of generating a formula or formulae of ingredients for a generated fragrance or fragrances, for user review.

S20 causes the computer to run a trained (or fine-tuned/retrained) palette generation ML model to generate at least one potential generated ingredient palette. Optionally, the generated palettes are for a specified fragrance end-use. The trained palette generation ML model is trained using the input dataset comprising ingredient palettes of known fragrances and/or flavours and ingredient concentrations of the known fragrances and/or flavours (and, optionally, end-uses of the known fragrances/flavours).

S22 causes the computer to run a trained concentration generation ML model to generate one or more potential generated formulae. The trained concentration generation ML model is trained using the input dataset. Optionally, this data is concatenated with one or more conditional variables, such as a specified end-use. Each generated formula comprises ingredient concentrations for one generated ingredient palette.

### Model Architecture—Palette Generation

The skilled reader will appreciate that many known generative models may be used for the generation of ingredient palettes.

As one worked example, a variational autoencoder, VAE, is shown to work successfully. A VAE is an AI algorithm, which is configured to encode and decode information. When encoding information, the VAE maps large amounts of information to smaller representations. This compressed representation of information is the latent space of the VAE, as the original information is hidden in this compressed representation. The decoder maps the latent space back to the original input. Unlike conventional autoencoders, the “variational” nature of VAEs means that VAEs employ variational (Bayesian) inference and learn probability distributions of input data. More specifically, the encoder outputs the mean and covariance corresponding to the posterior probability of given training data, and the decoder takes latent vectors—sampled from the output of the encoder—and reconstructs the sample data.

A modification that is shown to generate highly suitable prospective fragrances is that of a conditional variational autoencoder, CVAE. CVAEs are a variation of VAEs for cases where some label or group may be associated to each data point (say, a characteristic within a face) and conditional generation (by imposing some label) is the searched goal. The architecture of a CVAE is schematically the same as (or at least very similar to) that of a VAE, with the exception that the information of the label is shared with both the encoder and decoder.

In the following worked examples, the end-use of the fragrance (e.g., use in bleach products, use in perfume products, etc.) is imposed as the “condition”. Of course, alternative (or additional) conditions may be imposed, such as the author of the known fragrances in the BOM. In this case, the “style” of an expert author may be learnt by the CVAE and the CVAE, when trained, may be used to generate further fragrances in the style of a particular expert author.

CVAEs employed by the inventors are neural networks made of dense layers. The learning phase comprises encoding information carried by the training data (represented by formulae ingredients along with their concentrations) into a vector, which-when decoded-should return the initial formulae given to the model as an input.

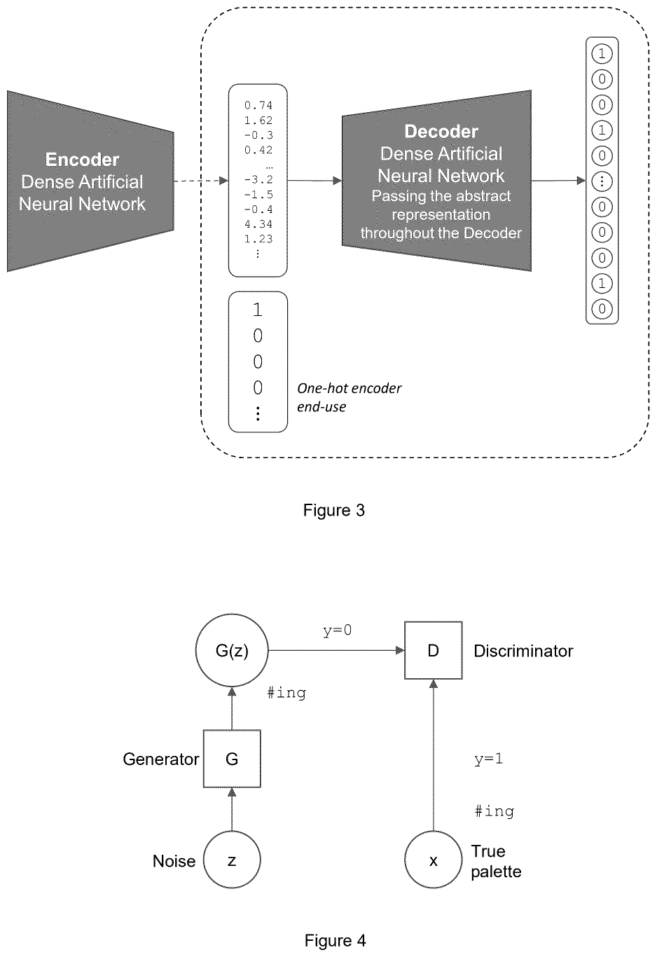

FIG. 3 is a schematic diagram, illustrating the generation of a palette with a trained CVAE.

The encoder outputs (to the decoder) a randomly sampled, abstract vector representation of formulae in the latent space (e.g., of dimensionality 100) via multivariate normal distributions, which parameters of which were learned by the CVAE during its training. Optionally, the intended end-use(s) of the formula may be concatenated to this latent space variable. The decoder outputs a reconstructed palette. For example, in the form of a one-hot encoded vector representation of the generated formula. Each dimension of this vector is associated with a specific ingredient in a catalogue of ingredient, where “0” means the ingredient is not used in the generated palette, and “0” means the ingredient is used in the generated palette.

Generative adversarial networks, GANs, are also shown to be well-suited to fragrance generation. GANs are a class of neural network architectures, introduced in Goodfellow et al. (2014). GANs are formed by two neural networks: a generator and a discriminator. Given samples from a low-dimensional known distribution (typically multivariate normal), the generator attempts to generate samples from the target distribution. The discriminator, on the other hand, attempts to discriminate between which samples are real (i.e., part of the training set) and which were generated by the generator. Thus, training a GAN essentially involves solving a minimax type problem, where the generator tries to fool the discriminator by generating new plausible examples from the problem domain, and the discriminator tries to classify examples as real (from the domain) or fake (generated).

This means that the generator is not trained to minimise the distance to a specific image, but rather to fool the discriminator. This enables the model to learn in an unsupervised manner.

More precisely, the two networks (generator/discriminator) are obtained by solving the following minimax problem, equation (1):

$${min}_{θ} {max}_{ϕ} V ( θ , ϕ ) = {E}_{x - {p}_{data} ( x )} [ log {D}_{ϕ} ( x ) ] + {E}_{z - p ( z )} [ log ( 1 - {D}_{ϕ} ( {G}_{θ} ( z ) ) ) ] \qquad ( 1 )$$

That is, to learn the generator's distribution p_{g} over data x, one may define a prior on input noise variables p_{z}(z), and represent a mapping to data space as G(z; θ_{g}), where G, generator, is a differentiable function represented by a multilayer perceptron with parameters θ_{g}. One may also define a second multilayer perceptron D(x; θ_{d}), discriminator, that outputs a single scalar. D(x) represents the score that x came from the data rather than p_{g}. One may train D to maximize the score of assigning the correct label to both training examples and samples from G. Simultaneously, one trains G to minimize log (1-D(G z))).

As mentioned in Arjovsky et al. (2017), there is a caveat when training these types of minimax problems: mode collapse/vanishing gradients. This issue generates instability of learning, especially when the discriminator is ahead of the generator. In order to circumvent these issues, the authors propose an alternative way to pose the problem by considering the Wasserstein distance between the original and the generated distribution. In more detail, for a so-called Wasserstein GAN, WGAN, the new minimax problem becomes problem 2:

$${min}_{θ} {max}_{ϕ} {E}_{{p}_{dαta}} [ {f}_{ϕ} ( X ) ] - {E}_{{p}_{z}} [ {f}_{ϕ} ( {G}_{θ} ( Z ) ) ] \qquad ( 2 )$$

There is an important technical detail in the previous formulation: the discriminator f_{φ} should be within the Lipschitz-1 function family. To fulfil this condition there are different regularization approaches, including weight clipping (which is not found to be very effective) and gradient penalty (Gulrajani et al. (2017)). When the target distribution is discrete, palette adaptations are required for better results. In the present case, the inventors have applied a Stochastic Presence transformation, where the principal idea is to transform the presence and absence (denoted by 1 and 0, respectively) of each ingredient within an ingredient palette to a continuous random representation. The stochastic transformation may be implemented through equality (3), which is applied for each ingredient independently:

$${T}_{γ} ( k ) := {( - 1 )}^{k + 1} ( {X}^{γ} / 2 + 0.5 ) \qquad ( 3 )$$

where X~U(0,1), k∈{0,1}, and 0<γ<1. This stochastic transformation may be seen as a pre-processing step, performed on original formulae at each epoch of the GAN process.

FIG. 4 provides a schematic overview of a GAN (and, equivalently, for a WGAN) for palette generation. The generator network, G, accepts noise, z, and generates a palette of ingredients, #ing, forming the generated palettes G(z). These generated palettes are allocated a label to indicate that they are generated (y=0), rather than real formulae palettes (y=1). The discriminator network, D, seeks to classify example palettes as real or fake (generated).

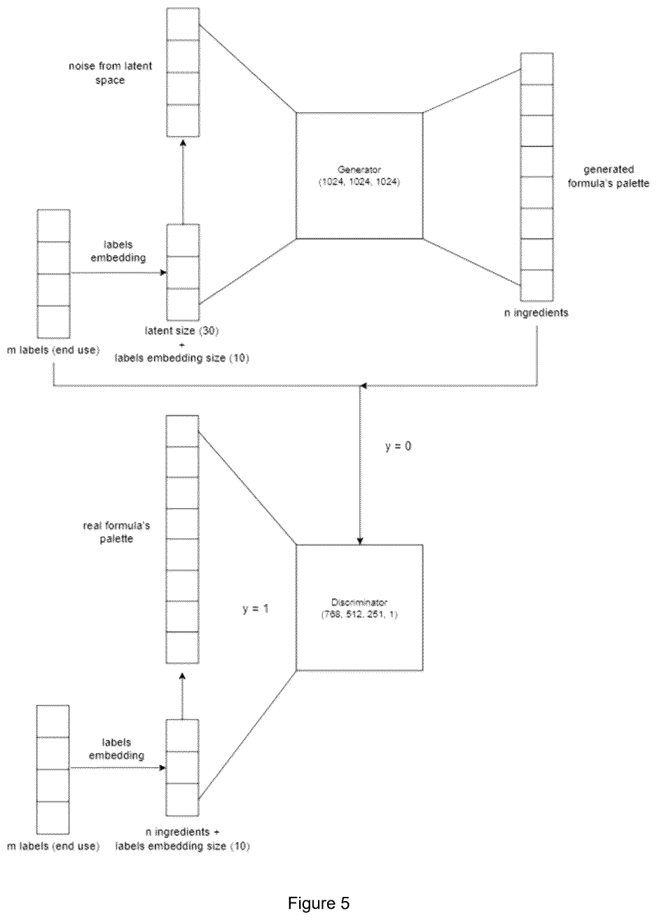

FIG. 5 is another schematic overview of an example GAN for palette generation. Noise from latent space representative of potential palettes (here, concatenated with end use) are passed through a generator network (of size 1024, 1024, 1024 in this example) to generate formulae palettes. The discriminator network (of size 768, 512, 251, 1 in this example) discriminates between these generated formulae palettes and real formulae palettes (here, also concatenated with end use).

### Model Architecture-Concentration Generation

The skilled reader will appreciate that many known generative models may be used for the generation of ingredient concentrations, following generation of ingredient palettes. The inventors have explored the use of a bucket predictor and the use of GANs.

In Zhang, Isola & Efros (“Colorful Image Colorization”, 2016), the authors deal with the computer vision problem of image colouring for black and white images. They propose to transform the regression problem into a multilabel classification task, by building buckets of “colour usage”, working on the principle that obtaining a rough estimate of the intensity of colour usage is sufficient to obtain realistic looking colouring. If an object may take on a set of distinct colour values, the optimal solution to the Euclidean loss will be the mean of the set. In colour prediction, this averaging effect favours grey-toned, desaturated results. The inventors have come to the realisation that similar techniques, using bucket predictors, BPs, are well suited for generating concentrations of ingredients (rather than “concentrations” of colours). That is, one may rephrase images as formulae, pixels as ingredients, and colours as the proportion of the ingredients. Observe that multimodality is also present for the ingredients' usages.

The BP is essentially an encoder-decoder for which the input is a one-hot encoded version of ingredients (i.e., palettes) and the output is, for each ingredient, the assignment of a bin on a logarithmic scale to which the ingredient's concentration belongs.

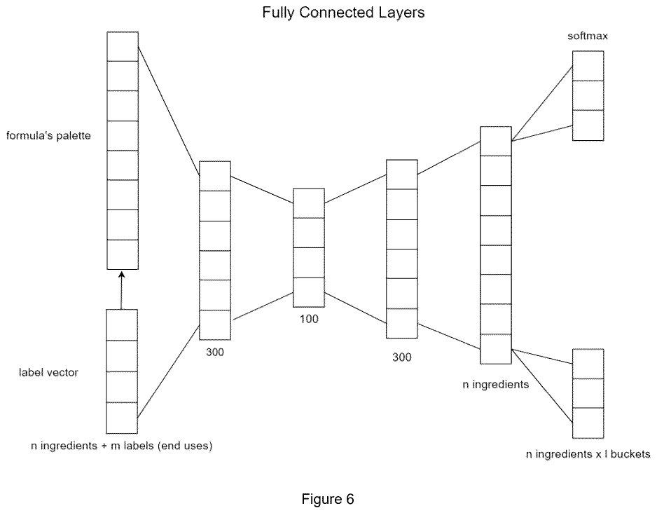

FIG. 6 is a schematic overview of an example BP for concentration generation. Formulae palettes, of size n_ingredients, are passed through a fully connected encoder-decoder architecture (the palettes may be concatenated with end use, in which case the size is, instead, n_ingredients+m_labels). The example BP encoder reduces the input via a layer of size 300 to a latent representation of size 100. The example BP decoder then increases the processed data to a size of 300 before increase to a size corresponding to the total number of ingredients. A softmax function is used as an activation function, normalising the output to a score representative of the distribution buckets of ingredients (e.g., a probability).

The inventors have demonstrated the use of GANs (e.g., WGANs) to assign ingredient concentration to generated palettes. As with BPs above, this approach has also been inspired by image colorization type of problems: in one example, the “noise” input comprises palettes drawn from the latent space, or—when trained—palettes previously generated by a GAN for palette generation, with the fragrance end-use concatenated.

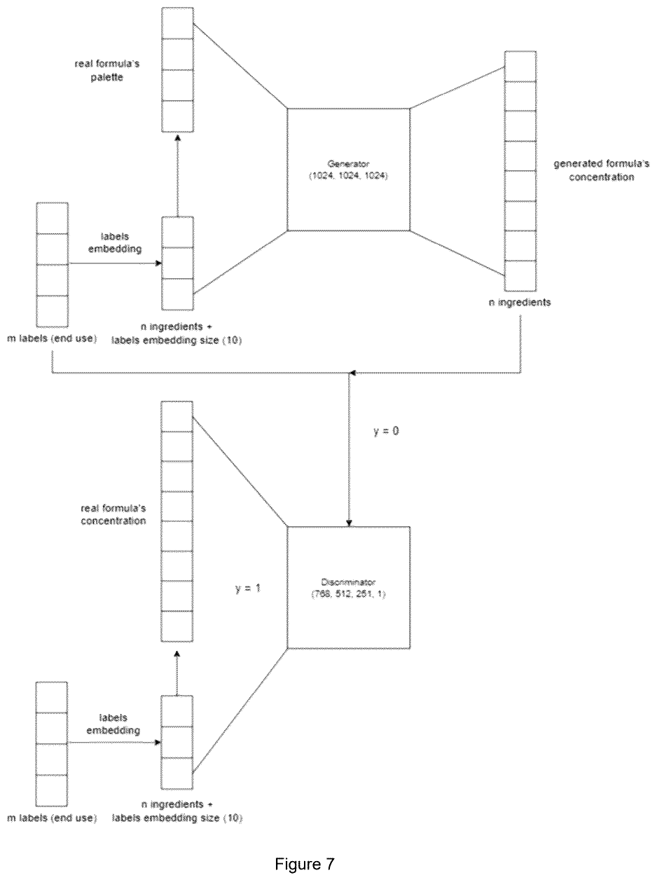

FIG. 7 is a schematic overview of an example GAN for concentration generation. Formulae palettes (here, concatenated with end use) are passed through a generator network (of size 1024, 1024, 1024 in this example) to generate formulae concentrations. The discriminator network (of size 768, 512, 251, 1 in this example) discriminates between these generated formulae concentrations and the real formulae concentrations (here, also concatenated with end use).

### Training Data

The skilled reader will appreciate that the specific structure of the training data may vary depending on the exact architecture and training process in use when implementing embodiments. In the present case, the inventors have utilised an internal bill of materials, BOM, which provides a data set of a collection of known formulae. Known formulae as those determined by experienced, expert perfumers.

The BOM data set has different levels of detail, where “level n” provides all individual raw materials, while “level 1” provides the ingredients and bases (mixtures of ingredients) that are available to perfumers. In the following examples, BOM level 1 has been utilised. In the present case, the BOM level 1 comprises the following schema:
group_code: a text variable that contains an identifying code of each formula.
ingredient_or_group_code: a text variable that contains the code for each ingredient.
concentration_over_100: float8 variable that is the concentration escalated to sum 100.

The BOM is provided in a CSV tabular format comprising approximately (after pre-processing) 150,000 known fragrances (but for example 5,000 or 50,000 fragrances could be used; the same applies to flavours) and each fragrance's constituent ingredients and each ingredient's concentration.

Specific steps involved in the pre-processing of the data prior to training for the worked example are as follows:
Rename all solvents to one generic name (the solvent in fragrances is merely a “carrier” for active ingredients; the non-active or odourless ingredients are removed to focus on the ingredients that provide the scent).
Remove functional ingredients (that is, ingredients, which do not provide any fragrance contribution, but are, instead, related to colour or texture for instance and may therefore be disregarded for the purposes of fragrance generation).
Remove formulae that use other formulae as ingredients.
Remove rare ingredients (ingredients used in fewer than 10 formulae).

More generally, pre-processing may be used to perform any or all of the following steps:
Selects valid formulae (e.g., those formulae for which: concentrations add up to 100 wt. %; identifying code format is in an expected form, e.g., the form of 3 letters followed by 3 numbers followed by 3 letters; formulae are non-recursive; formulae have sufficient ingredients to qualify as a formula).
Cleans ingredients (e.g., remove rare and odourless ingredients; gather all solvents as one general solvent; unify repeated ingredients; remove formulae that are left with an insufficient number of ingredients; renormalize, e.g., by wt. % when necessary; replace identifying group code with name when possible).
Creates features for ML model training (e.g., establish each formula as associated with an array of all the ingredients used in the BOMs and the ingredient concentrations, setting the former to zero if the ingredient is absent from the formula).
Splits data into train and test sets or train, test and validate sets (e.g., for the former, in a ratio 75:25, respectively, or, for the latter, 80:10:10, respectively).

Of course, where training data is already in a suitable format for training, some or all pre-processing steps may not be necessary.

In addition to the BOM, various tables of data may be collated to quantify generated formulae. For instance, in the present example, a table of odour descriptors for each ingredient may be used. Various systems of quantifying descriptor are available; in the present case a two-level approach was utilised, which describes ingredients by “family” and “characterizer” (stored as text variables in this example). An ingredient's family and characterizer(s) relate to olfactive characteristics of raw ingredient. Each raw ingredient may have one or two characterizers, typically assigned manually by experienced perfumers.

Those information at ingredient level may be used to derive a quantitative olfactive description of formulae (see below for description on an example implementation). In the present example, olfactive characteristics are used during the post processing phase of the pipeline (following formulae generation), to provide additional information. Of course, the skilled reader will appreciate that quantitative olfactive information may be used in the training process (alongside the corresponding ingredients).

Again in addition to the BOM, where formulae may be represented as ingredients and concentrations (for each formula there being as many rows as ingredients with their respective concentrations), other tables of data include tables of odourless ingredients (used in one example during pre-processing of the BOM), and tables of ingredient prices. The latter may be used to quantify generated formulae in terms of formula price. Again, the skilled reader will appreciate that in other examples, the price information may be used in the training process (e.g., alongside the corresponding ingredients).

### Model Architecture Modifications

### Frequency Thresholds

For generation using CVAE architectures/processes, each output unit represents the score (e.g., a number between 0 and 1) that the represented ingredient is present in the prospective palette. A straightforward approach for acceptance or rejection of the ingredient into the prospective formula is to consider that a value above 0.5 indicates presence. This approach, however, disregards the distribution of the presence of the ingredient within the training data.

Instead, the CVAE approach may be modified such that the presence threshold is specific to each ingredient in the palette: for each ingredient, the threshold value may be set to be equivalent to the frequency of appearance of the ingredient in the training set. With this approach, ingredients that appear less frequently in the training dataset should appear with the same frequency in the generated palettes. In contrast, with the static 0.5 threshold, these ingredients rarely appear in any generated palettes, despite having an (albeit rare) presence in training data.

### Condition Concatenation

In conditional models, the input typically comprises the formula and a label that provides contextual information (e.g., the end-use of the formula). By including this contextual information, the model may be trained with the intent that the model learns relationships between inputs with the same label. With many generative models, for instance with the CVAEs and bucket predictors used for palette generation and concentration generation, respectively, the contextual label may be used in the input of the model. To reinforce the contextual information about the input throughout the training process (and, in turn, throughout the inference process), the inventors have come to the realisation that one may reintroduce this contextual label at numerous processing stages of the underlying network (rather than just as input to specific modules, e.g., encoder and/or decoder).

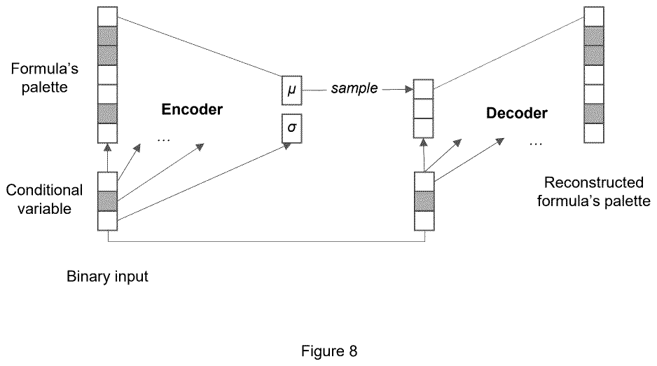

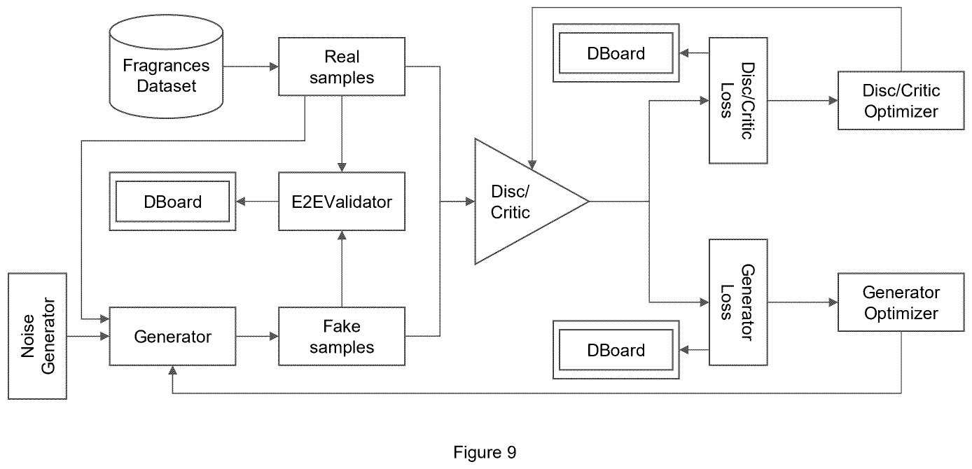

FIG. 8 is a schematic diagram, illustrating condition concatenation at each step of a CVAE architecture. The conditional variable (the end-use of the formula; represented here as a 3-value vector, where the shaded box indicates one-hot encoding of the end-use) is concatenated with the formula ingredient palette as input to the encoder block of the autoencoder architecture. At each layer of the encoder, the conditional variable is concatenated to the output of each layer of the neural network. Similarly, one the latent representation has been obtained by the encoder (represented with the mean and covariance corresponding to the posterior probability of given training data), the decoder block of the autoencoder architecture concatenates the conditional variable to the output of each later of the neural network. As shown in this simple diagram, the reconstructed formula palette matches that of the (original) formula palette (where, for the palettes, the blank boxes indicate the absence of an ingredient and the shaded boxes indicate the presence of an ingredient).

To summarise, the conditional label (e.g., the end-use) is one-hot encoded (i.e., converted into a vector filled with zeroes, except for one coordinate, which is assigned a value of “1” and which refers to an end-use depending on its position) and concatenated to the embeddings (abstract compressed representations) of the BOMs in the latent space of the CVAE.

The skilled reader will appreciate that condition concatenation may be applied, similarly, to GAN-based architectures.

### Batch Normalization

Batch normalization (Ioffe & Szegedy, “Batch Normalization: Accelerating Deep Network Training by Reducing Internal Covariate Shift”, 2015) is a technique that may be used to speed up a neural network's training process by normalizing each layer's output, without sacrificing model performance.

In the training process, for each mini-batch of inputs and for each layer, an activations matrix H is obtained and normalized into H′ so that each column is a normal distribution with mean 0 and variance 1. Before passing this information to the next layer, H′ may be reparametrized by the learned parameters γ and β, which represent the new variance and mean respectively:

$$γ {H}^{′} + β \qquad ( 4 )$$

These transformations have the effect that each layer's input is stable, helping the network's training process. The inventors have found that batch normalization techniques are well suited for improving the speed and stability of training processes for generative models in the generation of fragrance palettes and concentrations, including for CVAEs and GANs.

### Conditional GANs

As with conditional VAEs, discussed above, the inventors have recognised the benefits of enforcing conditions (i.e., the use case of the fragrance in this case) throughout the training and inference phases when using GAN architectures. These techniques consider a goal of allowing conditional generation, where the condition is fed to both the generator and discriminator. This variation (generally—not specifically in the field of fragrance generation) was proposed in Mirza & Osindero (“Conditional Generative Adversarial Nets”, 2014).

### Image Colorization Using GANs

In Nazeri et al. (“Image Colorization using Generative Adversarial Networks”, 2018), the authors make use of GANs for colorization of images. In this context, the “noise” distribution is that of grayscale images, while the target distribution is that of coloured images.

### Training

Conventional training techniques may be implemented in respect of each of the above-described example ML architectures (that is, for example, for VAEs/CVAEs/GANs for palette generation, and for BPs/GANs for concentration generation).

As an example, several architectures and hyperparameters were tested using BOM as training data, and using readily available GPUs and CPUs. Training of a WGAN for the generation of ingredient palettes with the example training dataset takes approximately 1 hour using a GPU. One model that performs well (where the model outputs are evaluated by means of a Random Forest classifier; see below in section entitled “Evaluation”) comprises the parameters as follows:
Model type: WGAN with Gradient Penalty
Latent space dimension: 30
#Layers: 5
#Units: [768,512,256]/[256, 512, 768].
Normalization: Batch Normalization but no regularization techniques.
Generator output activation: sigmoid (0,1)

Of course, other parameter values are suitable. For instance, as shown in FIG. 4 a generator with #Units [1024, 1024, 1024] and discriminator with #Units [768, 512, 512, 1] is suitable and works well.

FIG. 6 is a schematic overview of a training process for a GAN (and, equivalently, a WGAN) for palette generation. The fragrance dataset provides real samples, for use by the discriminator (disc/critic). The noise generator inputs noise into a generator network, which generates fake samples that are provided to the discriminator (disc/critic). During training of the discriminator network, the discriminator classifies both real data and fake data from the generator. The discriminator loss function (disc/critic loss) penalises losses incurred by the discriminator when misclassifying a real instance as fake or a fake instance as real. The discriminator updates its weights through backpropagation from the discriminator loss through the discriminator network.

The generator may be trained with the following procedure: sample random noise; produce generator output (i.e. a generated fragrance) from sampled random noise; obtain discriminator classification (real or fake) for generator output; calculate loss from the discriminator classification; backpropagate through both the discriminator and generator to obtain gradients; and use gradients to change only the generator weights. This is a single iteration of the generator training; the full GAN training process then alternates between training the discriminator for one or more epochs and training the generator for one or more epochs before desired convergence is obtained.

In this example case, data is output at various stages for user observation and assessment (via DBoard modules). In particular, end-to-end evaluation may is performed using real samples and fake samples; for instance, the module “E2EValidator” provides the out-of-bag, OOB, error computation, which is performed at each validation step to keep track of training. See below in the “evaluation” section for a discussion of this end-to-end evaluation.

Note that the schematic overview shown in respect of training a GAN is equally valid for the training process in respect of concentration generation if one were to replace the noise generator with a real palette (e.g., taken from the fragrance dataset).

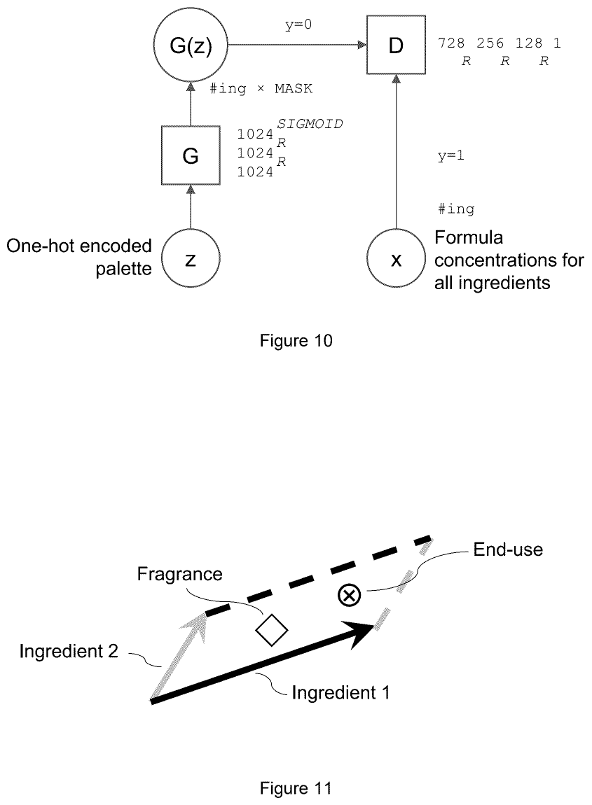

FIG. 10 is a schematic overview of a training process for a GAN (and, equivalently, a WGAN) for concentration generation. Here, a WGAN is used to assign concentrations to generated palettes. The noise input comprises palettes previously generated by a trained WGAN, with the fragrances end-use concatenated. The palette is described as a 0 to 1 vector of a dimension equivalent to the length of the available list of ingredients (in this case, approximately 1600).

The generator architecture is a multilayer perceptron, MLP, with dimensions [1024, 1024, 1024] and activation functions (between layers) [ReLU, ReLU, Sigmoid]. As some ingredients will have a positive concentration while not being present at the input, those ingredients are “muted” by using a mask (dependent on the input) after the last layer. The Generated concentrations (MASK) are based on the input one-hot encoded palette. Palettes with generated concentrations are assigned a label y=0.

The discriminator input comprises formulae with concentrations (real, with label y=1, and generated fake formulae). Real formulae are described by a vector, which coordinates are the concentrations of the formula's constituent ingredients. The discriminator comprises 3 hidden dense layers of sizes [728, 256, 128] intertwined with ReLU activation functions. The final layer of size [1] returns a score that indicates the label that the discriminator has assigned to each formula (i.e., fake or real).

Optimization of the discriminator and generator weights is performed as described above in respect of the GAN training process for the GAN for palette generation.

### Model Evaluation

Following successful generation of prospective formulae ingredient palettes, the inventors have evaluated the generated palettes in respect an expert perfumer's assessment of similarity to actual ingredient palettes. In respect of the VAE- and CVAE-generated palettes, the inventors have evaluated embeddings (latent representations) obtained by the trained encoder. Embeddings in this context are a high-level representation of the palettes, which stress particular details of the processed data.

In the developed evaluation techniques, the role of these representations may be expressed three-fold:
Exploring and/or understanding the information encoded by the palette-generating VAE, and being able to visually grasp different groupings formed by the VAE representations.
Allow visualization of the data distribution by the different generation algorithms previously proposed.
Allow identification of any relations between ingredients and fragrance end-uses, and relations between end-uses with one other.

That is, the driving questions behind the evaluation of generate palettes may be summarised as follows:
Is the VAE (or CVAE) representation able to distinguish end-uses? Otherwise, what other type of information is encoding?
Which part of the palette distribution is successfully covered with the palette generation techniques?

More conventional analysis techniques on latent representations using “classic” techniques, (such as: principal component analysis, PCT; t-distributed stochastic neighbour embedding, t-Sne; Random Projections, etc.), where clouds of points within latent representations may be plotted, were found not to provide any clear structure or teachings. For instance, assigning colours to each point according to some label (e.g., end-use), was not found to be useful in the context of evaluating generated palettes.

A first evaluation technique to this end, using latent representations, is based on the techniques proposed by Liu & Wang (“LatentVis: Investigating and Comparing Variational Auto-Encoders via Their Latent Space”, 2020). The authors propose to apply a linear transformation to the latent representation obtained by a VAE in order to grasp semantic directions of the data within the latent representation. The primary idea is to obtain this linear transformation by training a linear classifier that predicts some label (which defines a “semantic”) from the latent representation of the data. Afterwards, this linear transformation is used to define semantic directions in the latent space.

A second evaluation technique, instead working directly on palettes, uses a linear combination/convex embedding approach. The primary idea is to represent ingredients and end-uses as vectors in the plane (vᵢ)_{i′}(uₑ)ₑ∈R² in such a way that if a palette has proportions, (p)ᵢ, for the ingredient, i, with end-use, j, then:

$${p}_{1} {v}_{1} + … + {p}_{K} {v}_{K} = {u}_{l} \qquad ( 5 )$$

That is, the convex combination of the palette's ingredients results spatially close to the palette's end-use representation. In this way, one may obtain a representation for the palettes together with palette of ingredients and end-use.

FIG. 11 demonstrates this second evaluation technique schematically.

Notably, there are two related but distinct goals: generation (e.g., of ingredient palettes) and conditional generation (where the end-use is imposed for the generated palettes).

### General Generation

The performance of the generative models may be assessed by means of Random Forest classification, where the classifier is trained to distinguish between original and generated palettes. At this point, two possibilities have been considered and explored:
Use of the ingredients' presence or absence as the input for the classifier;
Use of bucketized versions of the palettes (buckets constructed from the proportions of each ingredient) as the input for the classifier. By doing this, the classifier will not be able to rely on fine details on the value of each concentration. That is, the classifier will not be able to rely on the bucket predictor output. Thus, in order to compare real and generated data, one needs to ensure that outputs are all expressed in the same way, meaning in buckets rather than in actual values of each concentration.

More precisely, the OOB score of the Random Forest classifier may be determined; the lower the OOB score, the better the generator is performing.

Additionally, for each generated palette, one may evaluate the palettes using the Jaccard index (or Jaccard similarity coefficient, distance, or intersection over union, IoU). For each generated palette, the lowest Jaccard distance to the training set of palettes is calculated as a metric for evaluation. This distance is based only on the presence or absence of ingredients.

### Conditional Evaluation

In order to evaluate how properly any conditional generator attains its goal (namely, generation of palettes for a specified end-use), the inventors have trained an end-use classifier on the original palettes. After generation, one may measure accuracy of the end-use predictions for generated palettes. Namely, if the classifier trained on original (real) data is able to properly predict the end-use of generated palettes (e.g., with high accuracy), then the generated palettes may be said to a have “proper end-use's trace”.

Here, the whole pipeline is evaluated in two different ways: a first (quantitative) manner, as described in the general generation section above (that is, using OOB scores for a fake/real classifier based on the bucketed palettes); and a second (more qualitative) manner. The second manner compares certain distributions from the real palettes with those obtained from the generated palettes. These are effectively “sanity-checks” and include production of:
Histograms, comprising of number of ingredients per palette; an
Box plots, for the number of ingredients per end-use.

Generally speaking, as quantified in terms of OOB score and in view of qualitative comments from experienced perfumers, conditional palette generators are found to be more effective than non-conditional generators.

TABLE 1 below demonstrates the OOB score and the accuracy of end-use classification for palettes, where generated palettes are generated using a CVAE. Various modifications to the underlying CVAE approach are explored: “T” denotes the use of frequency thresholds; “C” denotes the use of condition concertation (at various layers of CVAE architecture); and “B” denotes the use of batch normalization. Combinations of modifications are denoted with both (or all) relevant letters (e.g., “TB” or “TCB”).

TABLE 1
evaluation of palette generation capabilities of CVAE models

| Model (CVAE + | OOB score | Accuracy |
|---|---|---|
| modification(s)) | (real v. fake) | (end-use) |
| TCB | 0.651 | 0.194 |
| TC | 0.684 | 0.234 |
| TB | 0.686 | 0.139 |
| T | 0.661 | 0.176 |
| CB | 0.728 | 0.205 |
| C | 0.753 | 0.216 |
| B | 0.736 | 0.127 |
| None | 0.761 | 0.172 |

In terms of palette generation, all modifications are shown to provide an improvement in OOB score (that is, the classifier struggles to distinguish real palettes from generated palettes more so with palettes generated using the CVAE with TCB modifications than with palettes generated using the CVAE with no modifications). The most significant modification is shown to be use of frequency thresholds.

The combined modification of TC is shown to provide the most accurately classifiable generated palettes in terms of end-use. More generally, the use of condition concatenation (C) causes the generator to effectively respect the imposed end-use; this is consistent with the idea that sharing the information about the end-use across the different layers of the CVAE should increase “the memory” about this characteristic.

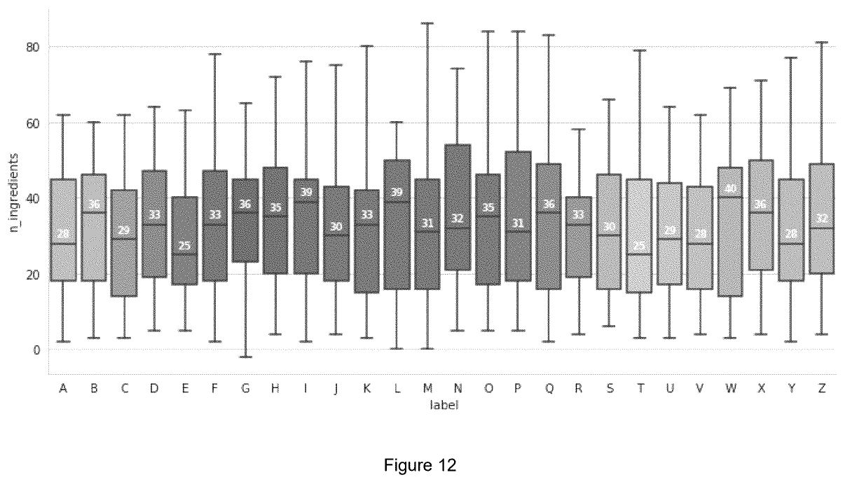

FIG. 12 is a box plot demonstrating the distribution of the number of ingredients for generated palettes across distinct end-uses (labelled A to Z), where the palettes are generated using a CVAE with the frequency threshold modification (CVAE+T). The OOB score here is 0.57; for reference a CVAE with no modifications imposed shows an OOB score of 0.66. Note that for this trial, the training dataset is distinct to that used to that used for the above-described trials (i.e., distinct BOMs are used). The distribution of ingredients per palette shows smaller palettes (in terms of the number of ingredients) than real distributions for most cases. There are, however, some atypical cases such as end-use E (which corresponds to bleach) that shows more ingredients than would be expected. An experienced perfumer indicated that the palettes look real, although the conditional end-use is not necessarily always respected.

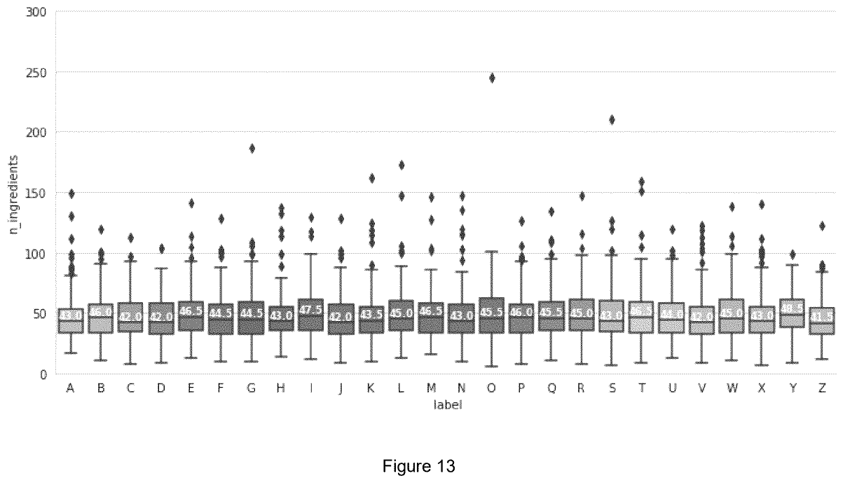

FIG. 13 is a box plot demonstrating the distribution of the number of ingredients for generated palettes across distinct end-use, where the palettes are generated using a CVAE with: the frequency threshold modification; the condition concatenation modification; and the batch normalization modification (CVAE+TCB). The OOB score here is 0.56. Relative to CVAE+T, the distribution of ingredients per palette is broader and results in more realistic patterns. For fine fragrances (end-uses M and W), the number of ingredients appears accurate; however, there are still larger, more unrealistic palettes for bleach (end-use E).

### Palette Size

As a further metric, the inventors have investigated the number of palettes to be generated in order to achieve a given subset of ingredients. This investigation is used to assess the completeness of the set of generated palettes using a GAN, although the findings are applicable to any generative model. Given a subset of ingredients {k₁, k₂, . . . , kₘ}, one may consider how many palettes should be generated to obtain at least one with the subset of ingredients. A simple estimate may be obtained by considering that each generated palette induces a Bernoulli variable: namely, the palette either contains or does not contain the subset of ingredients. This estimation requires one to estimate the following probability:

$$P ( {k}_{1} , … , {k}_{m} are present in generated palette ) ∼ \hat{p} ( {k}_{1} , … , {k}_{m} ) \qquad ( 6 )$$

With this estimation at hand, one may obtain a simple estimate of the minimum number of palettes to be generated, N(k), to obtain during the process—with a probability larger than β —at least one palette with the subset of ingredients:

$$N ( {k}_{1} , … , {k}_{m} ) \geq \frac{ln ( 1 - β )}{ln ( 1 - \hat{p} ( {k}_{1} , … , {k}_{m} ) )} \qquad ( 7 )$$

It is worthwhile to note that the problem of estimation of the aforementioned probabilities is somehow as difficult as the one of density estimation/generation. Further, for each subset of ingredients, there is a different N: the larger the size of the subset, the larger the estimated N. And finally, the estimation is highly dependent on the generated dataset; some subsets of ingredients will have better estimations than those subsets that are very unlikely.

There are two approaches to estimating N: firstly, a jointly empirical estimation, where one may calculate the ratio between the number of palettes (in the generated dataset) where the ingredients are present and J, the total number of generated palettes. And secondly, an empirical estimation for each ingredient, where, for each ingredient, k, one estimates the probability of this ingredient being present in a generated palette using the quotient between the number of palettes where ingredient k is present divided by J. Afterwards, and assuming that ingredients' presences are independent, one may bound the target probability by the product of the presence of probabilities for each ingredient in the subset. This approach provides a pessimistic bound because it does consider the existing correlation between ingredients.

With a generated dataset of J=250,000 palettes (10,000 for each of 25 possible end-uses), and for subset of four arbitrary ingredients (benzyl acetate, hedione, florhydral, and peach pure), the jointly empirical estimation approach provides an estimate of 173 palettes. The empirical estimation for each ingredient approach provides an estimate of 22,659,741 palettes.

Similarly, for the same generated dataset J, for five arbitrary ingredients (aubepine para cresol, yara yara, emonile, ebanol, galbanone 10) the jointly empirical estimation approach provides an estimate of 1,496 palettes. The empirical estimation for each ingredient approach provides an estimate of 24,071,494 palettes.

An experienced perfumer notes that the first approach (the jointly empirical estimation approach) provides numbers in accordance with “conventional” (i.e., not AI-supplemented) fragrance generation trials.

### Embedding Evaluation

Returning to the above-mentioned first evaluation technique (that is, using latent representations of the palettes; obtained by VAE or CVAE architectures in this case), the first evaluation technique is used to “guide” the two-dimensional representations of the VAE/CVAE embeddings by explicitly using end-use information. Namely, the representation is obtained by training a Linear Discriminant Analysis, LDA, classifier that predicts the end-use, and keeping the two principal components.

Even though the performance of this classifier is relatively poor in this case (accuracy: 0.51; with 14 end-uses to predict), this linear transformation of the latent representation is able to partially separate some end-uses. This means that part of the information when passed through the encoder is kept in the latent representation obtained by VAE.

Following a first implementation of the LDA, the inventors identified that modifications were possible. Firstly, it is possible to pre-process the proportions of use for each ingredient. Each ingredient has a different characteristic scale of use (i.e., each ingredient has a “proper” way of being used across palettes; for instance, some ingredients are typically used with relatively high concentration and other ingredients are usually used with relatively low concentration). However, what matters is the relative use of the ingredient with respect to its use within the whole dataset. Hence, instead of using the original proportions, the LDA may be modified to use

$${q}_{i} = {F}_{i} ( {p}_{i} ) \qquad ( 8 )$$

where Fᵢ is the empirical distribution for ingredient, i.

Secondly, it is possible to apply regularization terms for end-use representations. A first regularization term (statement (9)) may be used away from the origin with sufficient distance between end-uses: this prevents all representations from collapsing to the origin, and ensures that all end-uses “stick” to each other:

$$- {λ}_{A} {\sum }_{e} {\sum }_{{e}^{′}} {‖ {u}_{e} - {u}_{e}^{′} ‖}^{2} \qquad ( 9 )$$

A second regularization term (statement (9)) may be used to ensure that the representations are not too far away from the origin (else, the predominant term will be the previous term, and all representations will grow without barrier). This is therefore a way to control the relative distance of each end use from the origin; this ensures that all are roughly at the same distance from the origin:

$${λ}_{B} {\sum }_{e} {( ‖ {u}_{e} ‖ - 1 )}^{2} \qquad ( 10 )$$

Thus, the LDA may be modified for training with the following loss function (equation (10)):

$$L ( {\{ {v}_{i} \}}_{i ,} {\{ {u}_{e} \}}_{e} ) = {\sum }_{f} {( ‖ {q}_{1}^{f} {v}_{1} + … + {q}_{k}^{f} {v}_{k} - {u}_{{l}^{f}} ‖ - 1 )}^{2} {λ}_{B} {\sum }_{e} {( ‖ {u}_{e} ‖ - 1 )}^{2} - {λ}_{A} {\sum }_{e} {\sum }_{{e}^{′}} {‖ {u}_{e} - {u}_{e}^{′} ‖}^{2} \qquad ( 11 )$$

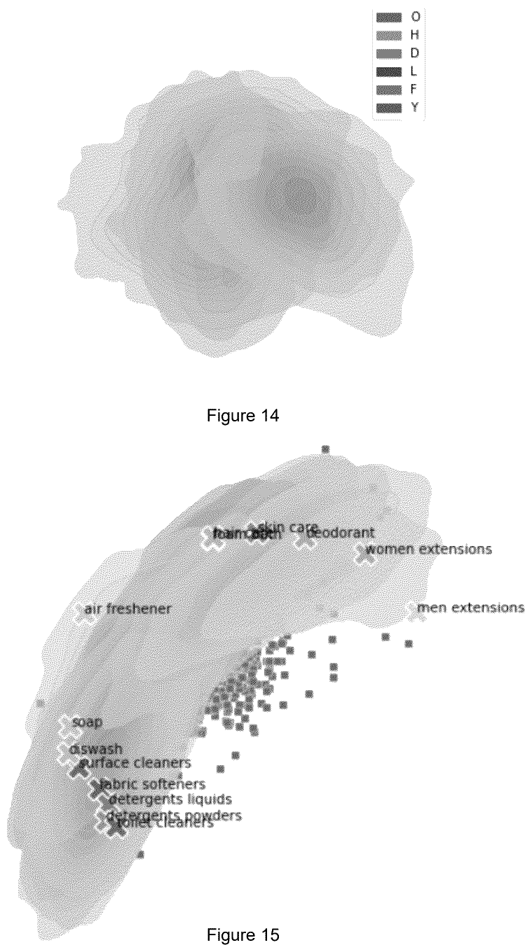

FIG. 14 is a two-dimensional KDE plot for the two principal components found for the embeddings, where the LDA uses the above modified loss function but does not involve pre-processing of the proportions of use for each ingredient. Plotted are representations of 6 distinct end-uses (O, H, D, L, F, Y). In this plot, crosses represent palette end-uses and dots represent ingredient within the palette embeddings (linear combinations of the palettes' ingredients). An experienced perfumer indicates that end-uses are “sticking”, according to the use of ingredients (generally speaking, ingredients are used more for specific end uses than others).

FIG. 15 is a two-dimensional KDE plot for the two principal components found for the embeddings, where the LDA uses the above modified loss function and involves pre-processing of the proportions of use for each ingredient. Qualitatively, these embeddings (and thus these generated palettes) are similar to those shown in FIG. 14.

To summarise, the above-described evaluation techniques show that palettes generated using a variety of generative ML models are similar to conventionally prepared fragrance palettes. In addition, generated palettes conform to expectations when an end-use is imposed (that is, for example, a generated palette for end-use A is difficult to distinguish from a known palette for end-use A). That is, palettes generated for specific end uses are distributed in a coherent way (relative to each other) in the latent space. In turn, AI-generated palettes are suitable for use in the manufacture of fragrances.

### Evaluation of Complete Formulae Generation (Palette and Concentration)

In addition to the above computation evaluation of generated palettes, the inventors have also investigated the results of complete formulae generation in a qualitative manner. That is, generated formulae (comprising generated palettes and accompanying generated concentrations) were compounded into actual fragrances. Generated formulae selected by perfumers for testing were sent to the creation software, from which perfumers can re-touch the formulae if necessary, check the formulae further, and generate and transmit instructions for compounding. The compounding is performed by a robot and, if necessary depending on the complexity of the mixing process, with the help of manual lab operators.

TABLE 2 below is a summary of evaluation results for 10 compounded generated formulae. The left-hand column indicates filters/searches applied to a large dataset of generated formulae. For example, with the top-most entry, the dataset was filtered to show formulae generated only for the end-use “fine fragrance women”, where the olfactive characteristics of the formulae were calculated to be of “family: fruity” and “characterizers: candied fruit, raspberry, blackcurrant” (see below for discussion of olfactive characterization of formulae). As shown in lower rows, other filters such as a maximum number of ingredients in the palette and the presence of particular ingredients within the palette may be employed. The middle column indicates the combination of models used to generate the formulae. For example, with the top-most entry, a GAN was used for the palette generation ML model, and a bucket predictor was used for the concentration generation ML model. VAER refers to a retrained VAE model. The right-hand column indicates the qualitative evaluation results from an experienced perfumer.

TABLE 2
evaluation of generated formulae

| Search | Model | Evaluation results |
|---|---|---|
| Fine fragrance Women, family: fruity. | GAN- | Nice fruity, rosy fresh blackcurrant, very good! |
| characterizers: candied fruit, | BP | |
| raspberry, blackcurrant | | |
| Fine fragrance Women, floral fruity, | VAE- | Powdery heliotropin and fruity, a real personal |
| max number of ingredients: 50, contain | BP | care impression but gently. An interesting idea, |
| undecavertol | | will need some retouch for more impact. |
| Fine fragrance Women, floral fruity, | VAE- | Beautiful sparkling citrus floral, a little |
| max number of ingredient: 50, contain | BP | technical/green/lily of the valley but it goes very |
| mahonial | | well. |
| Fine fragrance Women, floral fruity, | VAER- | Fruity, a little strawberry chewing gum, but an |
| max number of ingredients: 50, contain | BP | interesting jasmine floral base, a little specific to |
| mahonial | | musk. |
| Fine fragrance Women, floral fruity. | GAN- | Beautiful balanced fruity rose very pleasant, |
| Body spray idea. | GAN | natural green, no roughness and power. Surprised |
| | | not to feel the green notes and black pepper too |
| | | powerful. |
| Max number of ingredients: 15. | VAE- | Good first idea. Floral, powdery balsamic. Old |
| Contain ambrocenide. | GAN | rose. Interesting. To share with colleagues. |
| Hair care. Short idea. Max number of | VAE- | Beautiful patchouli rose idea, classic but |
| ingredients: 15, Families: Woody, not | GAN | overdosed rose ox overdosed. Interesting base to |
| aldehydic and no citronella. Contain | | develop. |
| ambrocenide. | | |
| Fine fragrance Men. Family: woody, | GAN- | Not bad! Smells balanced, oriental, quite classic |
| spicy, ambery, characterizer patchouli, | GAN | middle east, well done. A medicinal effect. |
| ingredients included: gaiacol and | | |
| ambrocenide | | |
| Families: Woody, Floral, Aromatic. | VAE- | Aromatic Fougere, more naturals than usual, nice |
| Exclude Earthy, Roasted. | GAN | smell, on the expensive side but good and |
| Characterizers: Amber dry, Apple, | | balanced masculine character, maybe for |
| Eucalyptus. | | softener, positively surprise by naturals impact, |
| | | junniper, cypres, etc. |
| Families: Floral, Green. Ingredients: | GAN- | Good impact hyacinth type, immediate interest for |
| Phenylacetaldehyde. Minimum number | GAN | detergents, Turkey for instance |
| of ingredients: 2, Max number of | | |
| ingredients: 25. | | |

### Worked Example

The inventors have trained at least five model (palette and concentration ML models) using the pandas Python software library.

Training data (in the format described above) is pre-processed as described above. That is, given a raw BOM of formulae, the pre-processing creates a table with cleaned data in the database. In addition, pre-processing for preparation of the data for use with Streamlit—an app framework, suitable for creating web abbs. The Streamlit processing creates olfactory descriptors for the original formulae (see below) and external schemas.

VAE and GAN palette generation models are trained to generate palettes of fragrances. The inputs for these two models are:
train_set (PandasFormulas): formulas used for training, as specified in catalog.yml under bom_train_formulas.
test_set (PandasFormulas): formulas used for validation, as specified in catalog.yml under bom_test_formulas
parameters (dict): hyperparameters of the model, present in parameters.yml

The outputs for these training processes are:
models: trained models are stored as pickle files as specified in catalog.yml under vae and gan_presence respectively
ingredients: list of all ingredients used, stored as a pickle file as specified in catalog.yml under ingredients
labels: list of all end-uses used. Stored as a pickle file as specified in catalog.yml under labels

BP and GAN concentration generation models are trained to, given a palette (the presence of certain ingredients) and an end-use, generate the concentration for each ingredient. Additionally, a Random Forest is trained and its OOB_score is stored to be used as an evaluation metric. The inputs for these models are:
train_set (PandasFormulas): formulas used for training, as specified in catalog.yml under bom_train_formulas.
test_set (PandasFormulas): formulas used for validation, as specified in catalog.yml under bom_test_formulas
parameters (dict): hyperparameters of the model, present in parameters.yml

The outputs for these training processes are:
models: trained models are stored as pickle files as specified in catalog.yml under bp and gan_concentration respectively
bp_oob: table with OOB_score as specified in catalog.yml under bp_oob
ingredients: list of all ingredients used, stored as a pickle file as specified in catalog.yml under ingredients
labels: list of all end-uses used. Stored as a pickle file as specified in catalog.yml under labels

Further, a CVAE retrained model is trained (or fine-tuned). In this case, this is a model comprising multiple VAE models, each retrained for a specific end-use. In order to run the CVAE model, the VAE model is be trained first. Additionally, a Random Forest is trained and its OOB_score is stored to be used as an evaluation metric. The inputs for this training process are:
model: stored VAE model as specified in catalog.yml under vae
train_set (PandasFormulas): formulas used for training, as specified in catalog.yml under bom_train_formulas.
test_set (PandasFormulas): formulas used for validation, as specified in catalog.yml under bom_test_formulas parameters (dict): hyperparameters of the model, present in parameters.yml

The outputs for this training process are:
vae_retrained: dictionary of end-use to its retrained VAE model. It is stored as a pickle file as specified in catalog.yml under vae_retrained
retrain_score: pandas dataframe with information about the retraining. For each end-use, it contains the OOB_score before and after the retraining. This is stored as a csv as specified in catalog.yml under retrain_score

For formulae generation, there are many model combinations that may be used. For each combination of models, there is a pipeline that is able to generate formulas (palettes and concentrations) by combining those models. In each pair of models, the first is used for palettes generation and the second for concentration assignment. For example:

### CVAE-BP and CVAE-GAN

Inputs:
   parameters (dict): parameters for generation, present in parameters.yml
   vae (CVAE): trained CVAE palette generator model.
   bp (FFPropPredictor): trained concentration assignment model (BP or GAN).
   train (PandasFormulas): formulas from training used for calculating Jaccard score.
   test (PandasFormulas): formulas from validation used for calculating Jaccard score.
   prices (PandasDataFrame): prices for each ingredient, taken from the DataBase.
Outputs:
   formulas (PandasDataFrame): In catalog.yml are specified the paths where the generated formulas are stored. For formulas generated with CVAE-BP, it is under formulas. For formulas generated with CVAE-GAN, it is under vae_gan_formulas
   formulas_info (PandasDataFrame): returns the Jaccard score, price, label and Mahalanobis distance for each formula, and the ‘git_commit’, ‘model_name’ and ‘date’ for each model. In catalog.yml are specified the paths where this information is stored. For CVAEBP, this is stored in the path defined in formulas_info. For CVAE-GAN, this is stored in the path defined in vae_gan_formulas

GAN-BP and GAN-GAN
Inputs:
   parameters (dict): parameters for generation, present in parameters.yml
   palette (GAN): trained GAN palette generator model.
   concentration_filler (GAN or BP): trained concentration assignment model
   train (PandasFormulas): formulas from training used for calculating Jaccard score.
   test (PandasFormulas): formulas from validation used for calculating Jaccard score.
   prices (PandasDataFrame): prices of ingredients to calculate final price of the formula.
Outputs:
   formulas (PandasDataFrame): In catalog.yml are specified the paths where the generated formulas are stored. For formulas generated with GAN-BP, it is under gan_bp_formulas. For formulas generated with GAN-GAN, it is under gan_gan_formulas.
   formulas_info (PandasDataFrame): all the information needed (Jaccard score, price and label) for each formula in addition to information about the model (git_commit, model name and date). In catalog.yml are specified the paths where this information is stored. For formulas generated with GAN-BP, it is under gan_bp_formulas_info. For formulas generated with GAN-GAN, it is under gan_gan_formulas_info.

CVAER-BP
Inputs:
   parameters (dict): parameters for generation, present in parameters.yml
   vaes (Dict[str, VAE]): Dictionary of VAEs model. Its keys are the end-uses for which the models were retrained.
   bp (FFPropPredictor): Model used to determine the concentration of the ingredients selected by the vaestest (PandasFormulas): Used to determine the name of the fragrances given their indices
Outputs:
   cvaer_bp_formulas: DataFrame that contains three columns (“formulas”: an integer that represents a created formula; “ingredient”: the name of the ingredient used in the formula; “concentration”: their concentration). It is stored in the path defined in cvaer_bp_formulas in catalog.yml
   cvaer_bp_formulas_info: returns the Jaccard score, price, label and Mahalanobis distance for each formula, and the ‘git_commit’, ‘model_name’ and ‘date’ for each model. The result is stored in the path defined in cvaer_bp_formulas_info in catalog.yml

In the above, the Mahalanobis distance for each formula is the Euclidean distance between the embedding of a generated formula of a given end-use and the centre (mean) of the region of the latent space covered by formulae of the same end-use.

After generating the formulae, the odour descriptors (for ‘family_1’ and ‘characterizer’) of each formula are computed.

After odour descriptors are computed, fragrances (original and generated) and associated information that are stored locally are uploaded to the DB.

To streamline this process, the inventors have implemented a Kedro pipeline structure, which is a framework for creating reproducible, maintainable and modular data science code. The whole process of generating formulas-pre-processing the data, training models, generating palettes, and then adding concentrations—may be represented using the Kedro-viz app.

The code may be divided in three parts:
1. Data pre-processing & Streamlit data preparation
2. Training AI models
3. Generating new formulas

In the present worked example, the Kedro pipeline are executed in the above given order.

### Olfactive Description of Formulae

Following generation of formulae (ingredient palettes and ingredient concentrations), techniques may be implemented to quantify the olfactive contribution of each formula.

The inventors have developed a technique for performing this function. In particular, the technique provides means for quantifying the fragrance family and fragrance characterizer (two manners of describing a fragrance) in terms of a numerical value. In summary, the technique involves: pre-processing of BOM data; rescaling concentrations into intensities; building an ingredient representation; representing an ingredient as a path in classification trees; projecting formula composition on the classification trees; computing formula similarity on each layer of the classification; aggregating layers into a global formula similarity; clustering formulae using K-nn label propagator; representing clusters for a user; and naming & querying the clusters. In one implementation, the technique is provided in a pandas-based Python framework.

Broadly, this technique is based on ingredient concentration statistics for each end use. For instance, for an end-use, if an ingredient's concentration is higher than typical, then one may conclude that the perfumer wanted to highlight this ingredient, and therefore the characterizer of the ingredient may be set to be one of the characterizers of the particular formula in question.

### Pre-Processing

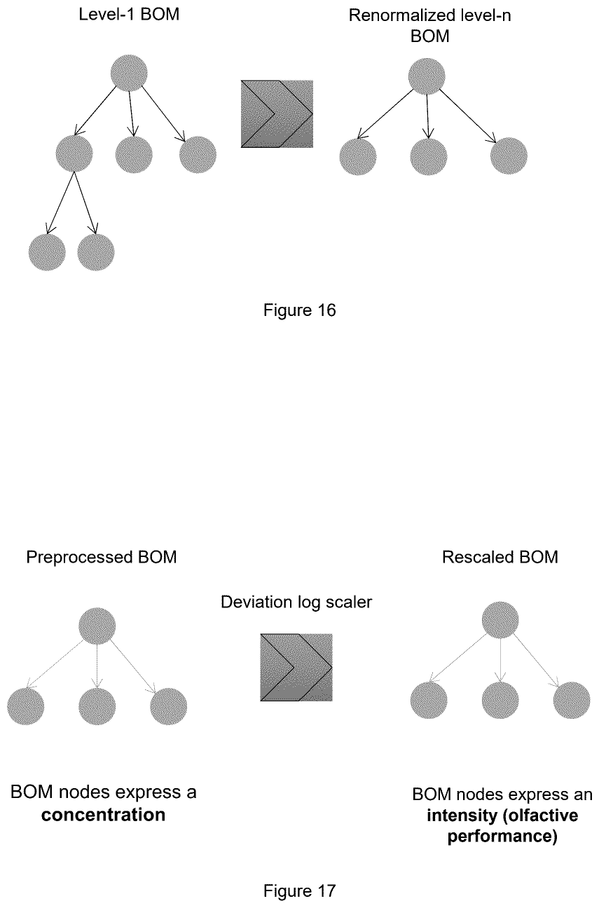

FIG. 16 is a schematic diagram of the effect of pre-processing for a level-1 BOM. Pre-processing obtains a renormalized level-n BOM. This pre-processing involves steps of:
Removing non-consumer product formulas, and removing “unconventional” end-uses (such as fine fragrances)
BOMs complete explosion (that is, transformation of the sub-formulae code within the BOM into a list of all ingredients included within the BOM)
Dealing with formulae featuring unclassified ingredients:
   Removing the ingredient when concentration is low enough
   Removing the formula otherwise
Removing technical (e.g., odourless) ingredients

Renormalizing to sum-up to 1 again (e.g., renormalizing the wt. % of the remaining ingredients, following any potential ingredient removal).

Rescaling Concentrations into Intensities

FIG. 17 is a schematic diagram illustrating the recasting of concentration (of ingredient) into intensity. This process involves:
Changing of the concentration to a base-3 log scale
Computing the mean and standard deviations on these log scaled concentrations
Computing the “deviation” from the mean, floored by 0.

This deviation may be interpreted as “how many standard deviation is the concentration above its mean?”. If concentration is below the mean, then the deviation is 0. The intensity refers to the added contributions of each ingredient of a formula to an olfactive characteristic. The contribution relies on the concentration of each ingredient in a formula and is compared to the average concentration of the same ingredient over all formulae, on a logarithmic scale. The idea is that an ingredient that has a higher dosage than usual in a formula will grant its olfactive characteristics to the formula as a whole.

Specifically, the following equation 11 is used to obtain the intensity form the concentration for each ingredient:

$$transform ( {p}_{j} ) = max ( 0 , \frac{log ( {p}_{j} ) - {μ}_{j}}{{σ}_{j}} ) : = {\overline{p}}_{j} \qquad ( 12 )$$

where μⱼ is the mean of log concentrations in which ingredient j is used (along the whole database, after the above-described steps) and σⱼ is the standard deviation of the log concentrations in which ingredient j is used. That is, now the formula is represented by ingredients and the transformed proportions (i₁, p̅₁), (i₂, p̅₂), . . . , (iₘ, p̅m). Note that the number of active ingredients may be less that in the original composition. Other transformations that achieve the same or similar effects are, of course, possible.

### Building an Ingredient Representation

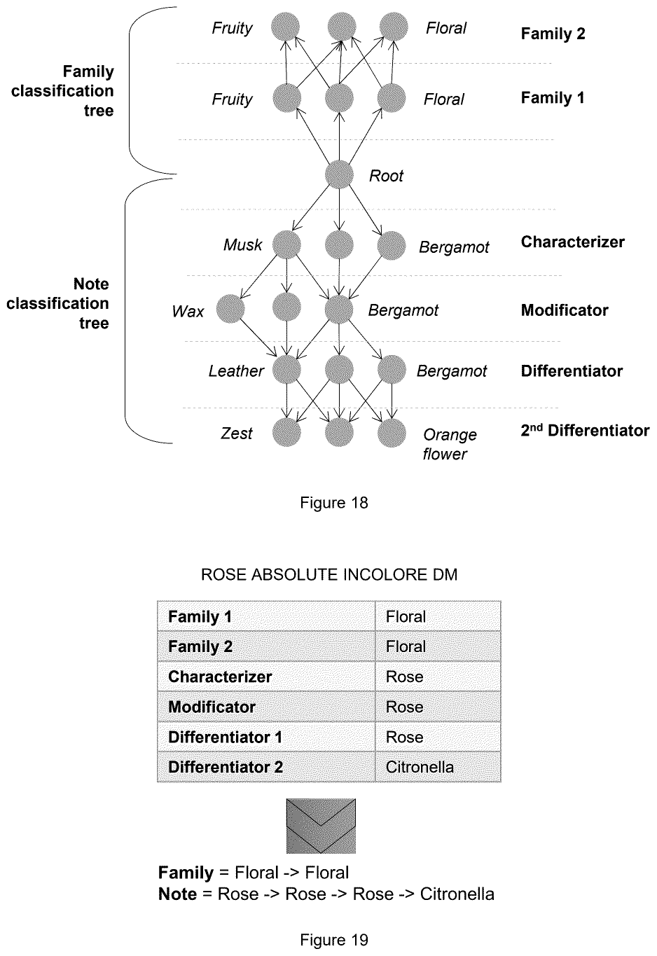

FIG. 18 is an example ingredient representation. The representation comprises a family classification tree and a note classification tree for a specific ingredient.

### Representing an Ingredient as a Path in Classification Trees

FIG. 19 is a schematic diagram illustrating the representation of an example ingredient (rose absolute incolore DM) as a path. For the ingredient representation, each ingredient is allocated a family and a note. These families and notes are represented as a path in the family and the note classification trees respectively. Canonical (or anchor) ingredients are those where all values along the note path are the same. In this example, the ingredient is very close to the canonical rose.

### Projecting Formula Composition on the Classification Trees

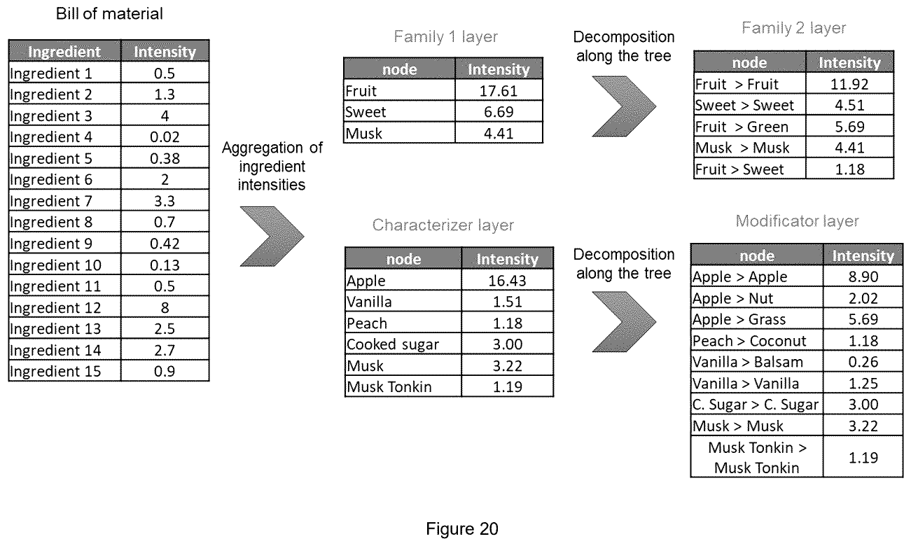

FIG. 20 is a diagram demonstrating the projection of an example formula into classification trees. The intensities of each ingredient are projected onto each layer of the tree so as to have representation of the formula at different granularities.

### Computing Formula Similarity on Each Layer of the Classification

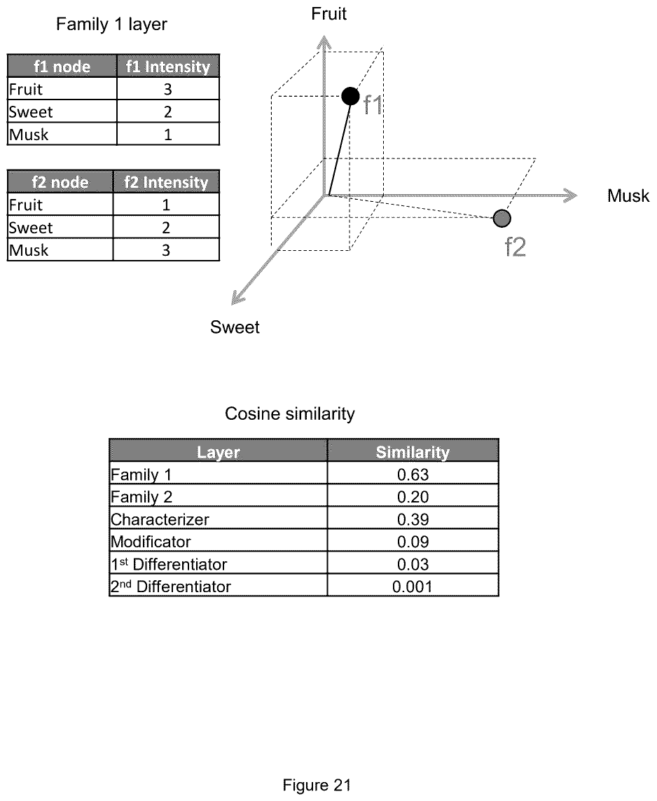

FIG. 21 is a diagram illustrating the computation of formula similarity, for each layer of classification. On each layer, the cosine distance corresponds to the angle between the vectors whose coordinates are the intensities on each possible node. The smaller the angle, the higher the similarity. The olfaction of two formulae will be considered close on a layer when the distance is small.

Aggregating Layers into a Global Formula Similarity

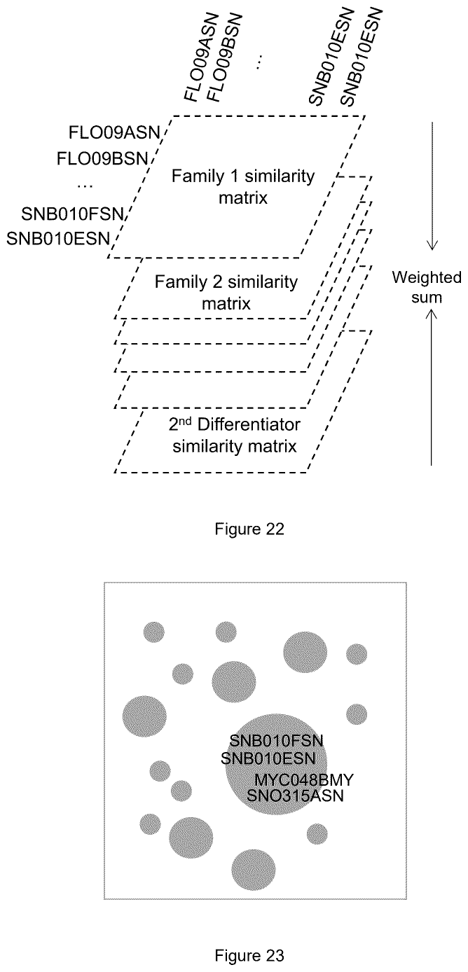

FIG. 22 is a diagram illustrating computation of the weighted sum of all layers. An aggregated pairwise similarity is computed between formulae by computing a weighted sum of distances at each granularity layer. The weights are a hyper-parameter of the model. With the above example of ML models for palette and concentration generation, only layers “family 1” and “characterizer” are employed; the weights for all other layers may be set to 0 to disregard these contributions.

### Clustering Formulas Using K-Nn Label Propagator

FIG. 23 is a diagram illustrating clustering of formulae. The K-nearest neighbour, K-nn, label propagator algorithm is applied to segment the pool of formulae into cluster (note that, e.g., SNB010FSN is an identifying name for one generated formula). The current number, K, of neighbours is set to 10; this hyperparameter may be adjusted to obtain a different cluster distribution if desired. In a worked example, the following numbers are observed:
Formula retained: 165,556
Number of clusters: 7,083
Minimum cluster size: 2
Maximum cluster size: 326
Mean cluster size: 23.37
Median cluster size: 17

### Representing Clusters for a User

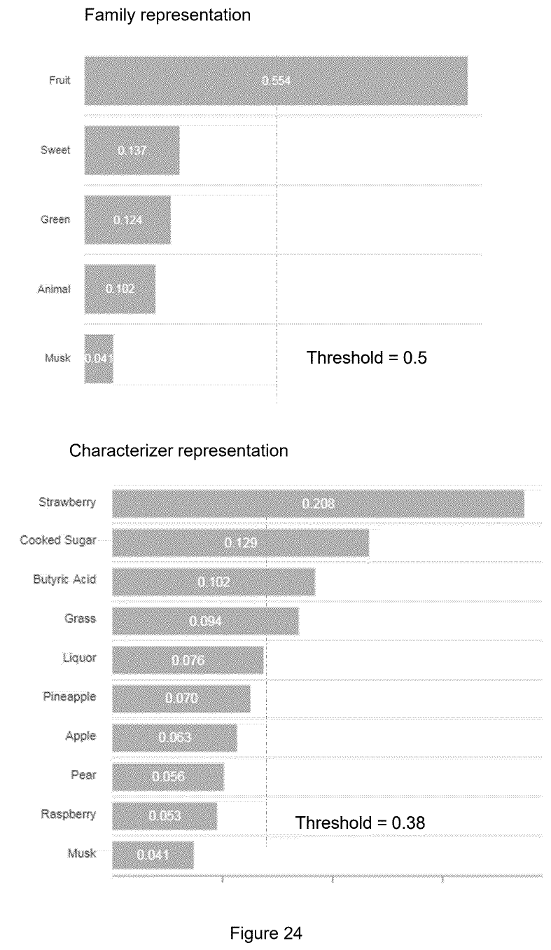

FIG. 24 is a pair of diagrams demonstrating the olfactive properties of an example formula, as determined with the above technique. The cluster characteristics are represented using the median intensities of formulae inside the cluster. The lists are truncated at a specific length: e.g., 5 for families, and 10 for notes (characterizer). This may of course be easily adapted.

### Naming & Querying the Clusters

The name of the clusters and the filtering criteria applicable to each cluster are extracted as a subset of the defined representation on both family and characterizer. All olfactive labels that have an intensity higher than a predefined fraction (i.e., a threshold) of the maximum intensity will be considered as a “naming” label. As seen in FIG. 24, considering a family threshold of 0.5 and a characterizer threshold of 0.38, the example formula may be described with the family “fruit” and with notes (characterizers) “strawberry”, “cooked sugar”, “butyric acid”, and “grass”.

In essence, the described technique for olfactive description of formulae involves mapping of a formula's palette and ingredient concentrations to predetermined olfactive values. This allows for quantitative clustering of a formula in respect of the olfactive contribution. In turn, this simplifies the user's assessment of formula—the user is provided with knowledge of the expected olfactive characteristics of a large number of formulae without the need for manufacturing the formulae.

### Graphical User Interface

Following generation of prospective formulae and, optionally, following olfactive characterisation of the generated formulae, a graphical user interface may be provided to the user to enable rapid and efficient observation and analysis of generated formulae.

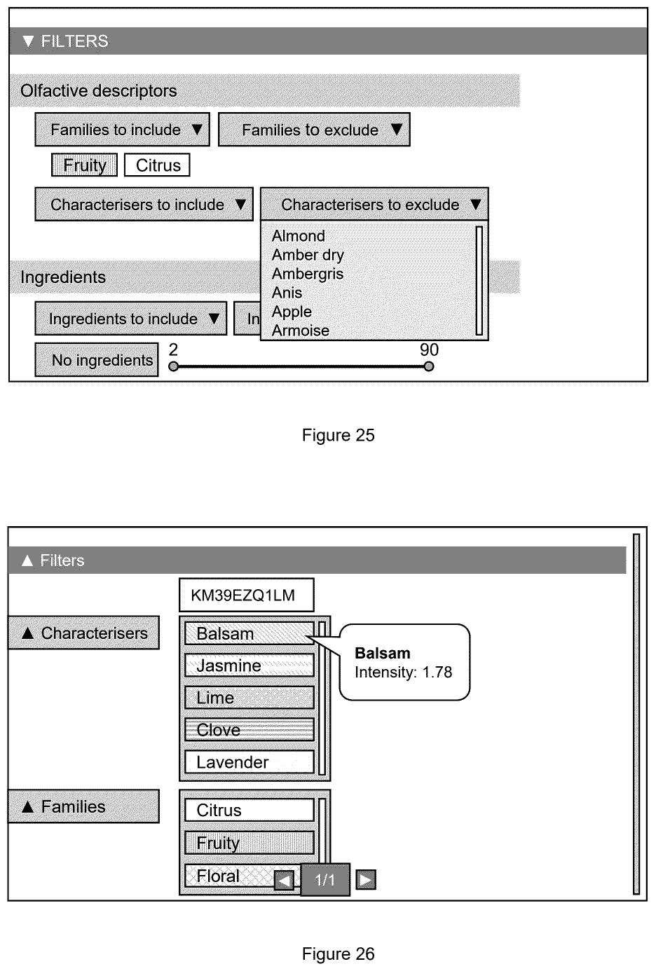

FIG. 25 is an example user interface, suitable for provision to a user to enable filtering of generated formulae. In this “filter” pane, the user is able to select a subset of formulae that conform to the user's preference. In the illustrated case, the user is filtering to show formulae that are classified within the olfactive families “fruity” and “citrus”. As shown, further filters may be applied, including exclusionary filters (“families to exclude”, “characterizers to exclude” “ingredients to exclude”), further inclusionary filters (“characterizers to include”, “ingredients to include”), and filters in terms of the numbers of ingredients present in formulae (here, any number between 2 and 90, inclusive). As shown, drop-down menus may facilitate the display of lists of data (in this case, characterizers). The skilled reader will appreciate that further filters (e.g., filtering by anticipated formula cost) may be implemented.

FIG. 26 is a further example user interface, illustrating the results of filtering. A subset of formulae that match the filter conditions as input by the user are shown (in this case, just a single formula meets the filter conditions). Characteristics of the formula “KM29EZQ1LM” are displayed, including the olfactive characterizers and olfactive families of the formula. On hover over a data point, further information is provided to the user. For example, as shown, on hovering over the characterizer “balsam”, a pop-up indicating that the calculated “balsam” intensity (calculated as per the olfactive description of formulae detailed above) is 1.78.

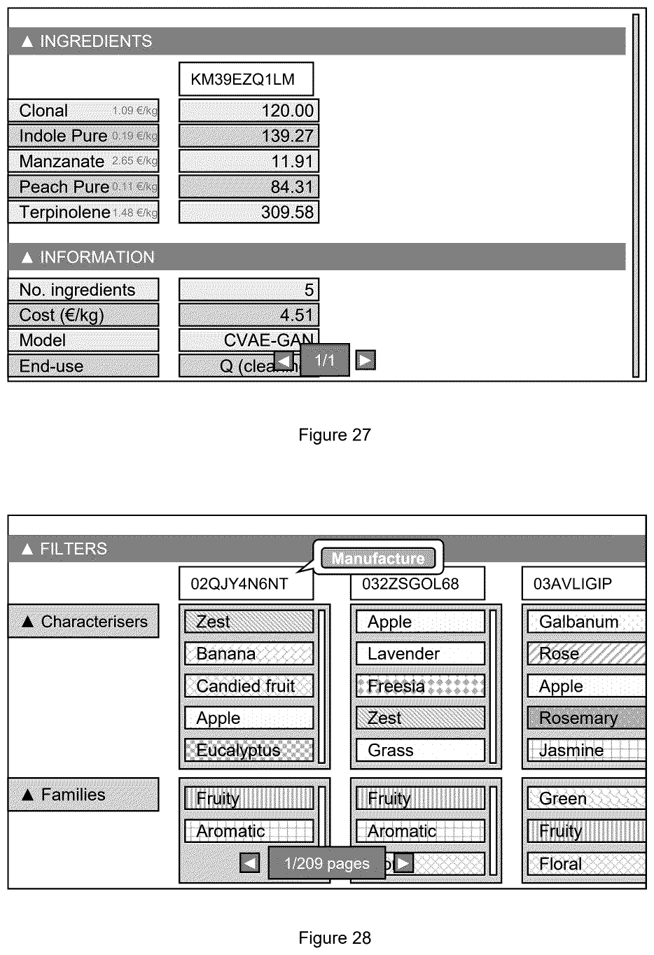

FIG. 27 is a further example user interface, illustrating further results of filtering. On scrolling, further information for the formula “KM29EZQ1LM” is provided to the user. As shown, this example formula comprises 5 ingredients (“clonal”, “indole pure”, “manzanate”, “peach pure”, and “terpinolene”) as the indicate concentrations. Further information is shown in the lower panel, including the expected price of fabricating the formula, where price information may be accessed from a separate table, on a per-ingredient basis. The models used for generation of the formula is also indicated (here, CVAE for palette generation, GAN for concentration generation). Finally, the anticipated end-use for the formula is indicated (here, “Q (cleaning)”).

FIG. 28 is another example user interface, illustrating the results of a distinct filtering process. As shown, the filtering parameters implemented here give rise to 209 pages of generated formulae.

Also as shown, the user interface provides functionality to enable the user to simply manufacture the desired formula. In this case, on selection of the “manufacture” button”, instructions may be generated for the manufacture of formula “02QJY4N6NT”. These instructions may be in the form of instructions for the user to follow (e.g., the quantity of each ingredient to compound). Alternatively, instructions may be in the form of instructions specifically tailored for transmission to a fragrance-manufacturing machine.

### Hardware

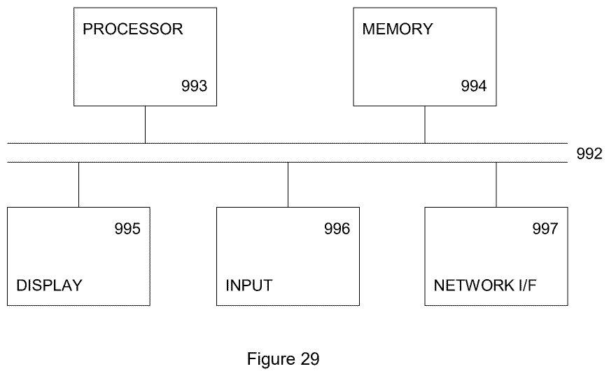

FIG. 29 is a block diagram of a computing device, such as a personal computer or a data storage server, which embodies the present invention, and which may be used to implement a method of an embodiment of generating a formula of ingredients for a fragrance and/or to implement a method of an embodiment of training a system for generating a formula of ingredients for a fragrance. The computing device comprises a processor 993, and memory, 994. Optionally, the computing device also includes a network interface 997 for communication with other computing devices, for example with fragrance manufacturing robots or machines.

For example, an embodiment may be composed of a network of such computing devices. Optionally, the computing device also includes one or more input mechanisms such as keyboard and mouse 996, and a display unit such as one or more monitors 995. The components are connectable to one another via a bus 992.

The memory 994 may include a computer readable medium, a term which may refer to a single medium or multiple media (e.g., a centralized or distributed database and/or associated caches and servers) configured to carry computer-executable instructions or have data structures stored thereon. Computer-executable instructions may include, for example, instructions and data accessible by and causing a general purpose computer, special purpose computer, or special purpose processing device (e.g., one or more processors) to perform one or more functions or operations. Thus, the term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device and execute processing operations, for example executing code stored in the memory to implement the various different functions of training modules and inference modules described here and in the claims. The memory 994 stores data being read and written by the processor 993. As referred to herein, a processor may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also include one or more special-purpose processing devices such as an application specific integrated circuit (ASIC), a field programmable gate array (FPGA), a digital signal processor (DSP), network processor, or the like. In one or more embodiments, a processor is configured to execute instructions for performing the operations and steps discussed herein.

The display unit 995 may display a representation of data stored by the computing device and may also display a cursor and dialog boxes and screens enabling interaction between a user and the programs and data stored on the computing device. The input mechanisms 996 may enable a user to input data and instructions to the computing device. For instance, the display unit 995 and input mechanisms 996 may enable a user to interact with the user interface suitable for filtering generated fragrances.

The network interface (network I/F) 997 may be connected to a network, such as the Internet, and is connectable to other such computing devices via the network. The network I/F 997 may control data input/output from/to other apparatus via the network. Other peripheral devices such as microphone, speakers, printer, etc. may be included in the computing device.

The computing device may comprise training processing instructions stored on a portion of the memory 994, the processor 993 being configured to execute the processing instructions, and a portion of the memory 994 being configured to store ML model training data during the execution of the processing instructions. For instance, the processor 993 may be configured to access training data (a BOM of fragrances) stored in memory 994 and implement the necessary forward- and back-propagation techniques to arrive at trained ML models. The trained models (including, e.g., details of node topology and trained weights) may be stored on the memory 994 and/or on a connected storage unit, and may be transmitted/transferred/communicated to a user for execution of the trained ML models.

The computing device may comprise generative processing instructions stored on a portion of the memory 994, the processor 993 being configured to execute the processing instructions, and a portion of the memory 994 being configured to store trained ML model data during the execution of the processing instructions. For instance, the processor 993 may be configured to access trained ML models stored in memory 994 and execute the trained models to arrive at generated palettes, generated ingredient concentrations, and/or generated formulae. The generated data may be stored on the memory 994 and/or on a connected storage unit, and may be transmitted/transferred/communicated to a user for, e.g., display or to, e.g., means for manufacturing the generated formulae.

Methods embodying the present invention may be carried out on a computing device such as that illustrated in FIG. 29 Such a computing device need not have every component illustrated in FIG. 29, and may be composed of a subset of those components. A method embodying the present invention may be carried out by a single computing device in communication with one or more data storage servers via a network. The computing device may be a data storage itself storing the trained fragrance generation ML models or generated formulae.

A method embodying the present invention may be carried out by a plurality of computing devices operating in cooperation with one another. One or more of the plurality of computing devices may be a data storage server storing at least a portion of the trained fragrance generation ML models or generated formulae.

1. A computer-implemented method of training a system for generating a formula of ingredients for a fragrance and/or a flavour, the method comprising:
receiving (S10) an input dataset comprising ingredient palettes of known fragrances and/or flavours and ingredient concentrations;
training (S12) a palette generation machine learning model using the input dataset, wherein the palette generation machine learning model, when trained, is configured to generate at least one generated ingredient palette; and
training (S14) a concentration generation machine learning model using the input dataset, wherein the concentration generation machine learning model, when trained, is configured to generate at least one generated formula, each generated formula comprising ingredient concentrations for a generated ingredient palette.

2. The method of claim 1, wherein the input dataset includes end-uses of the known fragrances.

3. The method of claim 2, further comprising:
receiving input of a specified end-use from amongst the end-uses of the known fragrances and/or flavours; and
training a retrained palette generation machine learning model by fine-tuning the trained palette generation machine learning model using the ingredient palettes and ingredient concentrations of the known fragrances and/or flavours associated with the specified end-use, and wherein training the concentration generation machine learning model uses ingredient palettes for the specified end-use.

4. The method of claim 1, wherein the palette generation machine learning model is a variational autoencoder, VAE, or a first generative adversarial network, GAN.

5. The method of claim 1, wherein the concentration generation machine learning model is a bucket predictor, BP, or a second GAN.

6. The method of claim 1, wherein:
the palette generation machine learning model, when trained, outputs scores indicating the presence of each ingredient within each generated ingredient palette, and
for each ingredient palette, the palette generational machine learning model is configured to determine that each ingredient is included within the ingredient palette when the score or presence of that ingredient exceeds a predetermined threshold, the predetermined threshold based on the presence of that ingredient in the known fragrances and/or flavours of the input dataset.

7. The method of claim 1, wherein:
the input dataset includes end-uses of the known fragrances and/or flavours;
the palette generation machine learning model is a VAE comprising an encoder-decoder architecture,
training the palette generation machine learning model comprises passing the end-use of the known fragrances and/or flavours to an encoder and a decoder of the palette generation machine learning model.

8. The method of claim 1, wherein:
the palette generation machine learning model is a VAE; and
training the palette generation machine learning model comprises, for each training mini-batch of the input dataset and for each layer, normalizing each layer output.

9. The method of claim 1, further comprising running the trained palette generation machine learning model and the trained concentration generation machine learning model to generate at least one generated formula.

10. The method of claim 9, further comprising computing olfactive descriptors for the at least one generated formula.

11. A computer-implemented method of generating a formula of ingredients for a fragrance, the method comprising:
running (S20) a trained palette generation machine learning model to generate at least one generated ingredient palette, the trained palette generation machine learning model trained using an input dataset comprising ingredient palettes of known fragrances and/or flavours and ingredient concentrations of the known fragrances and/or flavours; and
running (S22) a trained concentration generation machine learning model to generate at least one generated formula, the trained concentration generation machine learning model trained using the input dataset, wherein each generated formula comprises ingredient concentrations for a generated ingredient palette.

12. The method of claim 11, further comprising generating instructions to manufacture at least one formula in accordance with the corresponding ingredient palette ingredients and ingredient concentrations.

13. The method of claim 1, further comprising:
providing a user interface for displaying at least a subset of the at least one generated formula;
accepting user input into the user interface to select the subset of the at least one generated formula.

14. The method of claim 13, further comprising:
accepting user input into the user interface to cause manufacture of a formula.

15. A data processing apparatus comprising a memory and a processor,
the memory and the processor being configured for carrying out the method of claim 1.

16. A computer program comprising instructions which, when the program is executed by a computer, cause the computer to carry out the method of claim 1.

17. A computer-readable medium comprising instructions which, when executed by a computer, cause the computer to carry out the method of claim 1.
